(19) United States
(12) Patent Application Publication
(10) Pub. No.: US 2026/0165994 A1
(43) Pub. Date: Jun. 18, 2026

(54) TREATMENT OF LATE-ONSET NEURODEGENERATIVE DISEASES IN HETEROZYGOUS NPC1 GENE MUTATION CARRIERS

(71) Applicant: IntraBio Ltd., London (GB)

(72) Inventor: Michael STRUPP, Munich (DE)

(21) Appl. No.: 19/534,297

(22) Filed: Feb. 9, 2026

Related U.S. Application Data
(63) Continuation of application No. 17/758,759.
(60) Provisional application No. 62/960,637, filed on Jan. 13, 2020.

(51) Int. Cl. A61K 31/198; A61P 25/28

(52) U.S. Cl. CPC A61K 31/198; A61P 25/28

(57) ABSTRACT

The present disclosure provides methods of treating, preventing, or delaying the onset of a late-onset neurodegenerative disease, e.g., Niemann-Pick type C, or a symptom thereof in a subject in need thereof, comprising administering a therapeutically effective amount of acetyl-leucine to the subject, wherein the subject is heterozygous NPC1 gene mutation carrier.

### BACKGROUND OF THE INVENTION

### Field of the Invention

The present disclosure provides methods of treating, preventing, or delaying the onset of a late-onset neurodegenerative disease in a subject in need thereof, and/or treating, preventing, or delaying the onset of one or more symptoms of a late-onset neurodegenerative disease in a subject in need thereof, comprising administering a therapeutically effective amount of acetyl-leucine to the subject, wherein the subject is a heterozygous NPC1 gene mutation carrier.

### Background

Niemann-Pick type C (NPC) is an autosomal recessive lysosomal storage disease affecting an estimated 1:100.000 people. The disease presents with cerebellar ataxia, vertical and subsequently horizontal supranuclear saccade and gaze palsy, dystonia, pyramidal features, dysarthria, dysphagia, seizures, cognitive decline, and hepatosplenomegaly (Patterson 1993). Early-infantile, late-infantile, juvenile (31%) and adolescent/adult-onset (27%) forms occur. A positive family history of late-onset neurodegenerative disease such as Alzheimer's dementia, amyotrophic lateral sclerosis, Parkinson's disease (PD), or multiple system atrophy is common in NPC families, reported by (50.9% or 29 of 57 families) (Kresojevic et al. 2014).

NPC is metabolically and clinically closely related to Gaucher's disease (GD) where homozygosity causes a complex metabolic disease, while heterozygosity predisposes to late-onset neurodegeneration. Indeed, heterozygous glucosidase beta acid (GBA) mutations are the strongest known genetic risk factor for developing late-onset (GD) (Sidransky et al. 2009) Similarly, NPC heterozygosity may manifest clinically with a neurodegenerative disorder (Josephs et al. 2004; Harzer et al. 2014; Kluenemann et al. 2013), and genetic sequencing studies revealed NPC1 mutations in cohorts of adults with dementia or parkinsonism (Cupidi et al. 2017; Zech et al. 2013). Plasma metabolic profiles from heterozygotes are also abnormal (Probert et al. 2017). NPC heterozygosity occurs with a carrier frequency of 1:200 in the general population. But there are no systematic clinical studies of NPC heterozygotes, and it remains unknown to what degree heterozygosity may predispose a subject to late-onset neurodegeneration. Bremova-Ertl et al., Neurology April 2020, 94 (16) e1702-e1715.

There exists a need in the art to treat, prevent, or delay the onset of late-onset neurodegenerative diseases, e.g., NPC; and/or to treat, prevent, or delay the onset of one or more symptoms associated with late-onset neurodegenerative diseases, e.g., NPC, in heterozygous NPC1 gene mutation carriers (referred to herein as NPC heterozygotes).

### BRIEF SUMMARY OF THE INVENTION

Applicant unexpectedly discovers that acetyl-leucine can be administered to NPC heterozygotes to treat, prevent, or delay the onset of NPC; and/or treat, prevent, or delay the onset of one or more symptoms associated with NPC. Thus, in one aspect, the present disclosure provides methods of: (i) treating, preventing, or delaying the onset of a late-onset neurodegenerative disease; (ii) treating, preventing, or delaying the onset of one or more symptoms of a late-onset neurodegenerative disease; or (iii) treating, preventing, or delaying the onset of a late-onset neurodegenerative disease and preventing or delaying the onset of one or more symptoms of a late-onset neurodegenerative disease, comprising administering an effective amount of acetyl-leucine to a human subject in need thereof, wherein the subject is a heterozygous NPC1 gene mutation carrier. In another aspect, the subject is asymptomatic for the late-onset neurodegenerative disease at the time the initial dose of acetyl-leucine is administered to the subject. In another aspect, the late-onset neurodegenerative disease is NPC. In another embodiment, the acetyl-leucine is acetyl-L-leucine.

It is to be understood that both the foregoing summary and the following detailed description are exemplary and explanatory only, and are not restrictive of the invention as claimed.

### DETAILED DESCRIPTION OF DRAWINGS

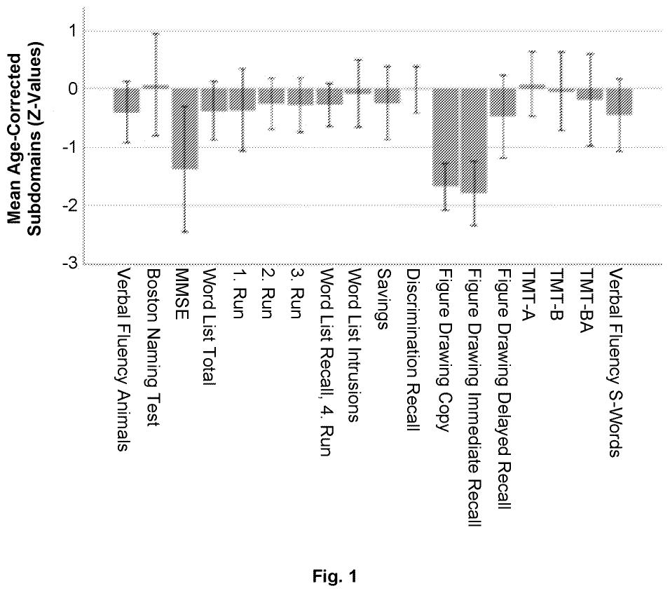

FIG. 1 is a bar graph showing the results of neuropsychological testing in the battery proposed by the Consortium to Establish a Registry for Alzheimer's Disease (CERAD) in heterozygous NPC1 gene mutation carriers.

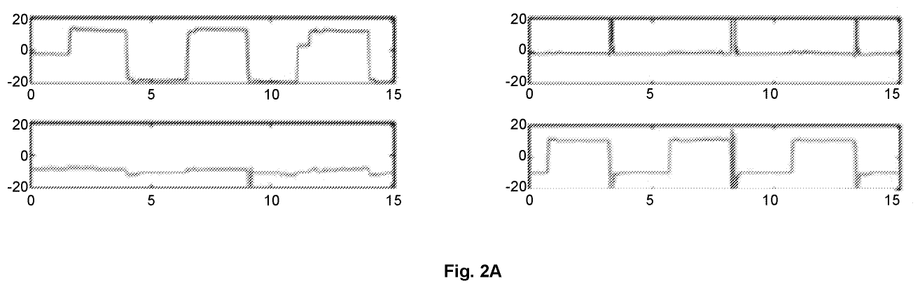

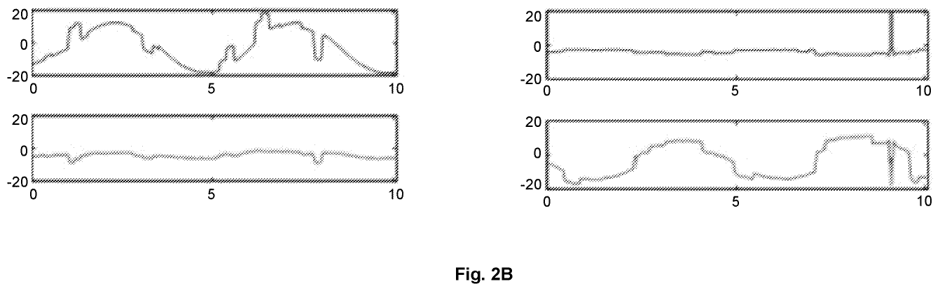

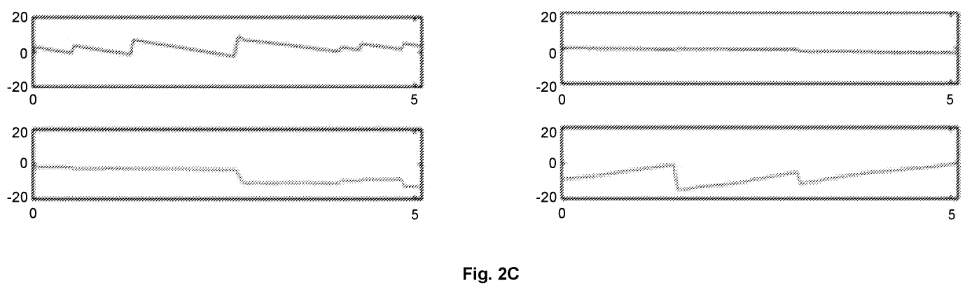

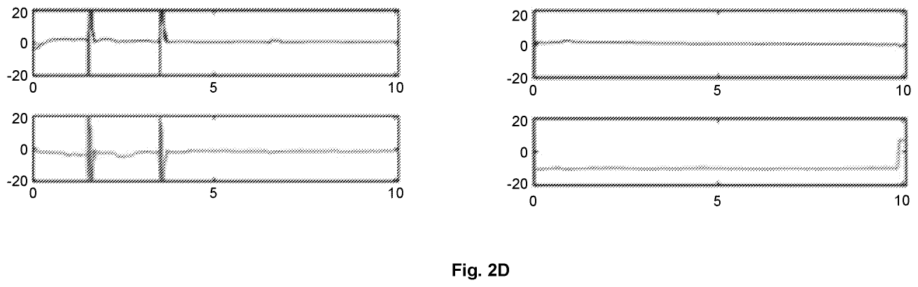

FIG. 2A is a line graph showing representative ocular motor findings with respect to 20° reflexive downward saccades in a heterozygous NPC1 gene mutation carrier. In the left-hand graphs the subject performs horizontal reflexive saccades, and in the right-hand graphs the subject performs vertical reflexive saccades. The upper panels indicate horizontal eye movement, and the lower panels indicate vertical eye movement. For example, this study shows that horizontal saccades are almost intact and that peak velocity of vertical saccades downward is lower than that of upward saccades.

FIG. 2B is a line graph showing representative ocular motor findings with respect to 30° reflexive saccades in a heterozygous NPC1 gene mutation carrier. In the left-hand graphs the subject performs horizontal reflexive saccades, and in the right-hand graphs the subject performs vertical reflexive saccades. The upper panels indicate horizontal eye movement, and the lower panels indicate vertical eye movement.

FIG. 2C is a line graph showing representative ocular motor findings with respect to horizontal and vertical self-paced saccades in a heterozygous NPC1 gene mutation carrier. In the left-hand graphs the subject performs horizontal reflexive saccades, and in the right-hand graphs the subject performs vertical reflexive saccades. The upper panels indicate horizontal eye movement, and the lower panels indicate vertical eye movement

FIG. 2D is a line graph showing representative ocular motor findings with respect to gaze holding nystagmus in 10° upgaze in a heterozygous NPC1 gene mutation carrier. In the left-hand graphs, the subject performs horizontal reflexive saccades, and in the right-hand graphs, the subject performs vertical reflexive saccades. The upper panels indicate horizontal eye movement, and the lower panels indicate vertical eye movement

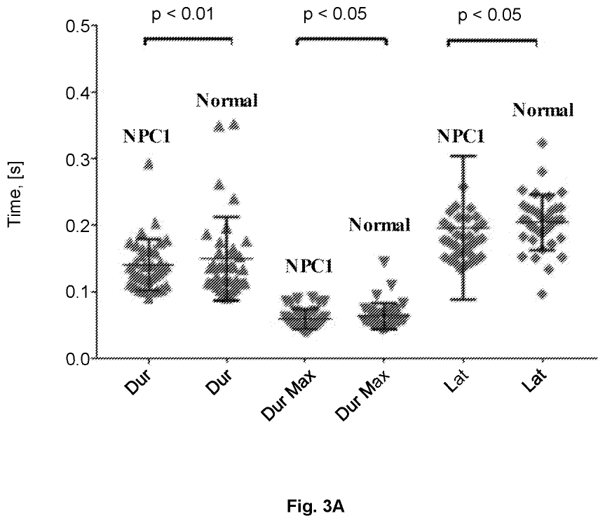

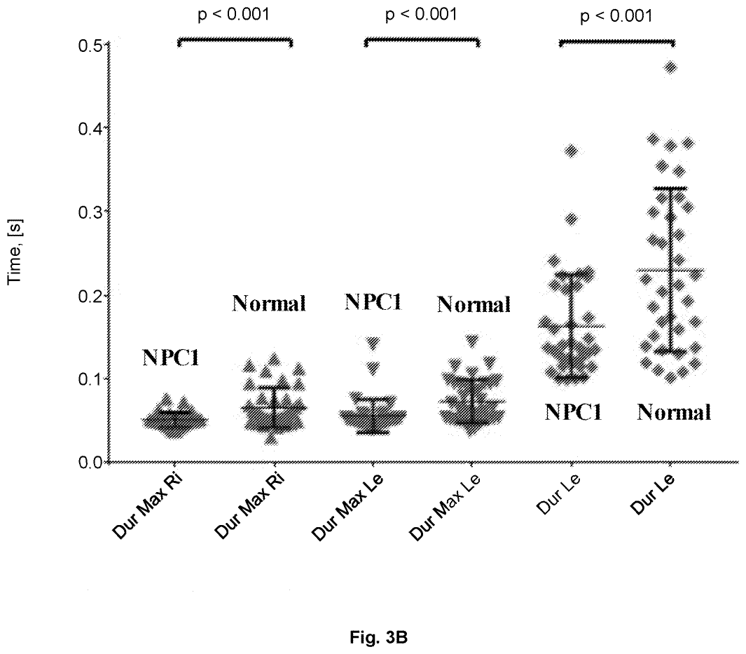

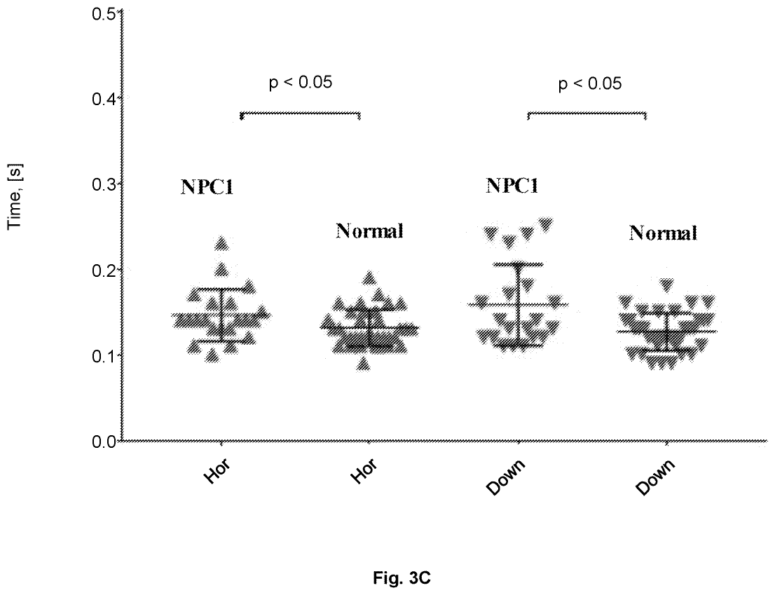

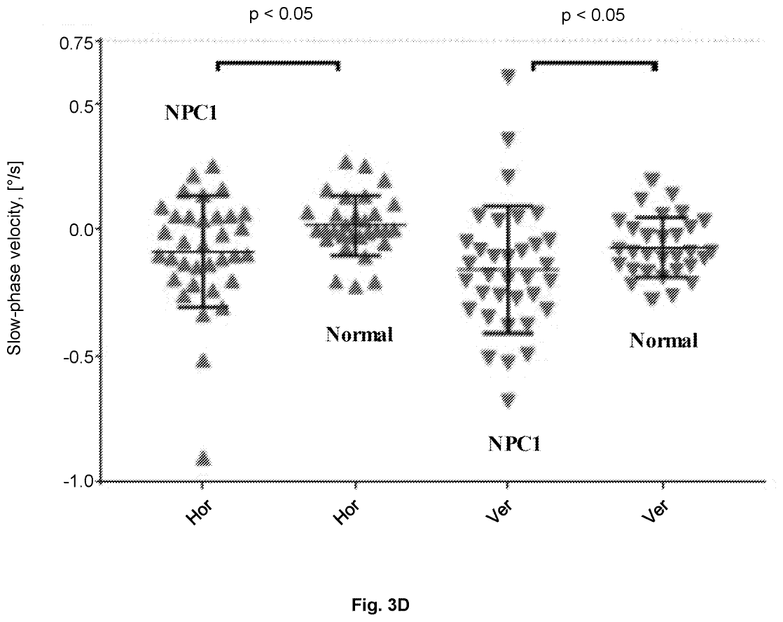

FIG. 3A is a scatter graph showing the mean duration (“Dur”), duration to reach peak velocity (“Dur Max”) and latency of 20° reflexive downward saccades in heterozygous NPC1 gene mutation carriers (“NPC1”) as compared to a normal control subjects (“Normal”).

FIG. 3B is a scatter graph showing the mean duration (“Dur”) and duration maximal (“Dur Max”) both rightward (“Ri”) and leftward (“Le”) of 30° reflexive saccades in heterozygous NPC1 gene mutation carriers (“NPC1”) as compared to a normal control subjects (“Normal”).

FIG. 3C is a scatter graph showing the mean duration of horizontal (“Hor”) and vertical (“Down”) of self-paced saccades in heterozygous NPC1 gene mutation carriers (“NPC1”) as compared to a normal control subjects (“Normal”). FIG. 3D is a scatter graph showing the mean slow-phase velocity of horizontal (“Hor”) and vertical (“Ver”) gaze-evoked nystagmus in 100 upgaze in a heterozygous NPC1 gene mutation carriers (“NPC1”) as compared to a normal control subjects (“Normal”).

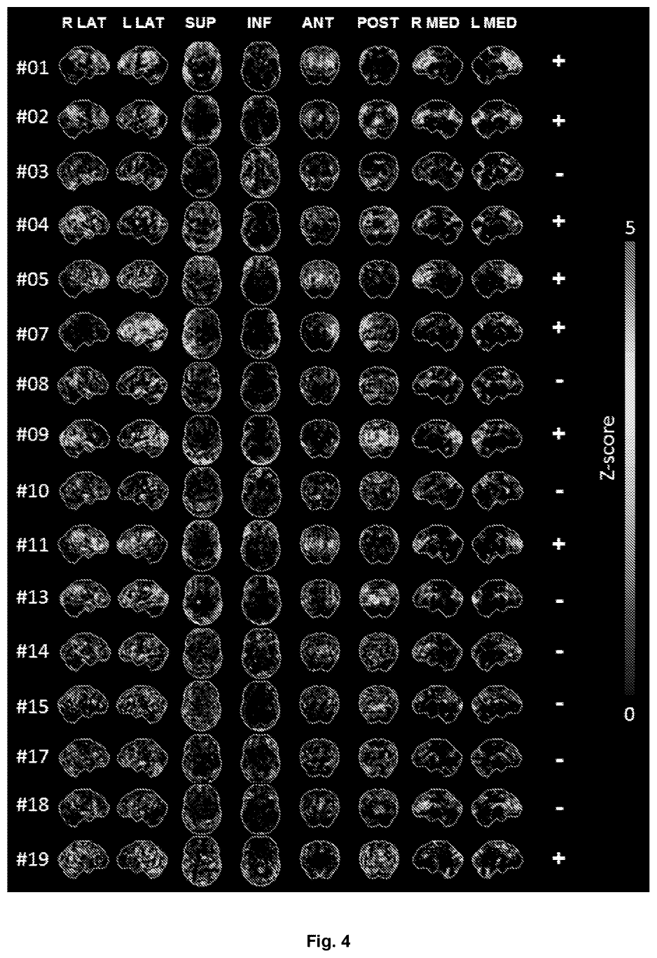

FIG. 4 is an image showing the stereotactic surface projections with individual hypometabolism patterns of NPC heterozygote subjects studied with brain FDG PET. Colored regions indicate less glucose metabolism as compared to age matched controls. The binary read of a significant pattern of neuronal injury is given as +=positive/−=negative.

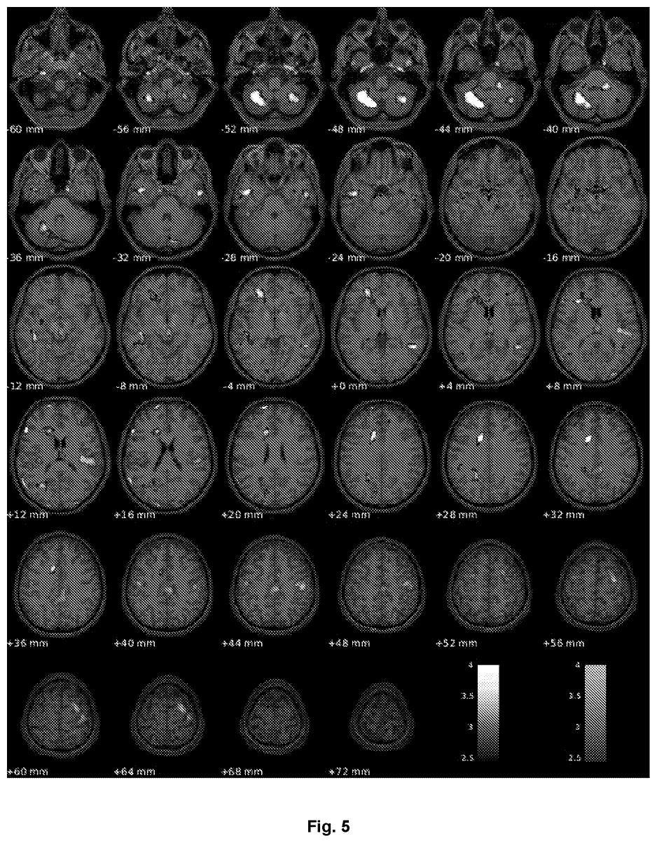

FIG. 5 is an image showing voxelwise brain FDG PET analysis in NPC heterozygote subjects.

### DETAILED DESCRIPTION OF THE INVENTION

In one embodiment, the present disclosure provides a method of treating, preventing, or delaying the onset of a late-onset neurodegenerative disease and/or one or more symptoms thereof, comprising administering a therapeutically effective amount of acetyl-leucine to a human subject in need thereof, wherein the subject is a heterozygous NPC1 gene mutation carrier. This is referred to as “Embodiment 1.”

The disclosure provides the following particular embodiments related to Embodiment 1.

Embodiment 2. The method of Embodiment 1, wherein the subject has any one or more of (a) an elevated level of chitotriosidase in blood or plasma; (b) an elevated level of cholestane-3β,5α,6β-triol in blood or plasma; (c) oculomotor abnormalities; or (d) hepatosplenomegaly.

Embodiment 3. The method of Embodiments 1 or 2, wherein the subject is asymptomatic for the late-onset neurodegenerative disease at the time of the initial administration of the acetyl-leucine.

Embodiment 4. The method of any one of Embodiments 1-3 for preventing or delaying the onset of the late-onset neurodegenerative disease.

Embodiment 5. The method of Embodiment 4 for delaying the onset of the late-onset neurodegenerative disease.

Embodiment 6. The method of any one of Embodiments 1-5, wherein the late-onset neurodegenerative disease is Niemann-Pick type C, Alzheimer's disease, amyotrophic lateral sclerosis, Parkinson's disease, or dementia.

Embodiment 7. The method of Embodiment 6, wherein the late-onset neurodegenerative disease is Niemann-Pick type C.

Embodiment 8. The method of Embodiment 6, wherein the late-onset neurodegenerative disease is Alzheimer's disease.

Embodiment 9. The method of Embodiment 6, wherein the late-onset neurodegenerative disease is amyotrophic lateral sclerosis.

Embodiment 10. The method of Embodiment 6, wherein the late-onset neurodegenerative disease is Parkinson's disease.

Embodiment 11. The method of Embodiment 6, wherein the late-onset neurodegenerative disease is dementia.

Embodiment 12. The method of any one of Embodiment 1-3 for preventing or delaying the onset of one or more symptoms of the late-onset neurodegenerative disease.

Embodiment 13. The method of Embodiment 12 for delaying the onset of one or more symptoms of the late-onset neurodegenerative disease.

Embodiment 14. The method of Embodiments 12 or 13, wherein the late-onset neurodegenerative disease is Niemann-Pick type C, Alzheimer's disease, amyotrophic lateral sclerosis, Parkinson's disease, or dementia.

Embodiment 15. The method of Embodiment 14, wherein the late-onset neurodegenerative disease is Niemann-Pick type C.

Embodiment 16. The method of Embodiment 15, wherein the one or more symptoms comprise cerebellar ataxia, dysarthria, dysphagia tremor, epilepsy, vertical supranuclear palsy, sleep inversion, gelastic cataplexy, dystonia, spasticity, hypotonia, ptosis, microcephaly, psychosis, progressive dementia, progressive hearing loss, bipolar disorder, or depression.

Embodiment 17. The method of Embodiment 14, wherein the late-onset neurodegenerative disease is Alzheimer's disease.

Embodiment 18. The method of Embodiment 17, wherein the one or more symptoms comprise difficulty performing familiar tasks, memory loss, disorientation to time and place, loss of good judgment, problems with abstract thinking, misplacing things, rapid mood swings, sudden and dramatic personality changes, loss of initiative, sleeping longer than usual, or loss of interest in usual activities.

Embodiment 19. The method of Embodiment 14, wherein the late-onset neurodegenerative disease is amyotrophic lateral sclerosis.

Embodiment 20. The method of Embodiment 19, wherein the one or more symptoms comprise muscle weakness or atrophy, spasticity, trouble swallowing or breathing, cramping, or slurred and nasal speech.

Embodiment 21. The method of Embodiment 14, wherein the late-onset neurodegenerative disease is Parkinson's disease.

Embodiment 22. The method of Embodiment 21, wherein the one or more symptoms comprise tremor, bradykinesia, muscle stiffness, impaired posture and balance, loss of automatic movements, speech changes, or writing changes.

Embodiment 23. The method of Embodiment 14, wherein said late-onset neurodegenerative disease is dementia.

Embodiment 24. The method of Embodiment 23, wherein the one or more symptoms comprise memory loss, difficulty communicating or finding words, difficulty with visual and spatial abilities, difficulty reasoning or problem-solving, difficulty handling complex tasks, difficulty with planning and organizing, difficulty with coordination and motor functions, confusion, or disorientation.

Embodiment 25. The method of any one of Embodiments 1-24, wherein 1 gram to 30 grams of acetyl-leucine are administered to the subject per day.

Embodiment 26. The method of any one of Embodiments 1-25, wherein the acetyl-leucine is administered to the subject in combination with another therapeutic agent.

Embodiment 27. The method of any one of Embodiments 1-26 wherein acetyl-DL-leucine is administered to the subject.

Embodiment 28. The method of any one of Embodiments 1-26 wherein acetyl-L-leucine is administered to the subject.

Embodiment 29. The method of any one of Embodiments 1-28, wherein the NPC1 gene mutation is a c.3246-25A>G mutation.

Embodiment 30. The method of any one of Embodiments 1-28, wherein the NPC1 gene mutation is a c.3246-5_3246-7del mutation.

Embodiment 31. The method of any one of Embodiments 1-28, wherein the NPC1 gene mutation is a c.2660C>T mutation.

Embodiment 32. The method of any one of Embodiments 1-28, wherein the NPC1 gene mutation is a c.2861C>T [S954L] mutation.

Embodiment 33. The method of any one of Embodiments 1-28, wherein the NPC1 gene mutation is a c.2861C>T mutation.

Embodiment 34. The method of any one of Embodiments 1-28, wherein the NPC1 gene mutation is a c.2776G>A mutation.

Embodiment 35. The method of any one of Embodiments 1-28, wherein the NPC1 gene mutation is a c.3010T>C mutation.

Embodiment 36. The method of any one of Embodiments 1-28, wherein the NPC1 gene mutation is a c.2474A>G mutation.

Embodiment 37. The method of any one of Embodiments 1-28, wherein the NPC1 gene mutation is a c.2978delG mutation.

Embodiment 38. The method of any one of Embodiments 1-28, wherein the NPC1 gene mutation is a c.3245+1dup mutation.

Embodiment 39. The method of any one of Embodiments 1-28, wherein the NPC1 gene mutation is a c.1211G>A mutation.

Embodiment 40. The method of any one of Embodiments 1-28, wherein the NPC1 gene mutation is a c.1843C>T mutation.

Embodiment 41. The method of any one of Embodiments 1-28, wherein the NPC1 gene mutation is a c.3182T>C mutation.

In another embodiment, the present disclosure provides acetyl-leucine for use in treating, preventing, or delaying the onset of a late-onset neurodegenerative disease and/or one or more symptoms thereof, wherein the subject is a heterozygous NPC1 gene mutation carrier. This is referred to as “Embodiment I.”

The disclosure provides the following particular embodiments related to Embodiment I.

Embodiment II. The acetyl-leucine for use of Embodiment I, wherein the subject has any one or more of (a) an elevated level of chitotriosidase in blood or plasma; (b) an elevated level of cholestane-3β,5α,6β-triol in blood or plasma; (c) oculomotor abnormalities; or (d) hepatosplenomegaly.

Embodiment III. The acetyl-leucine for use of Embodiments I or II, wherein the subject is asymptomatic for the late-onset neurodegenerative disease at the time of the initial administration of the acetyl-leucine.

Embodiment IV. The acetyl-leucine for use of any one of Embodiments I-III for preventing or delaying the onset of the late-onset neurodegenerative disease.

Embodiment V. The acetyl-leucine for use of Embodiment IV for delaying the onset of the late-onset neurodegenerative disease.

Embodiment VI. The acetyl-leucine for use of any one of Embodiments I-V, wherein the late-onset neurodegenerative disease is Niemann-Pick type C, Alzheimer's disease, amyotrophic lateral sclerosis, Parkinson's disease, or dementia.

Embodiment VII. The acetyl-leucine for use of Embodiment VI, wherein the late-onset neurodegenerative disease is Niemann-Pick type C.

Embodiment VIII. The acetyl-leucine for use of Embodiment VI, wherein the late-onset neurodegenerative disease is Alzheimer's disease.

Embodiment IX. The acetyl-leucine for use of Embodiment VI, wherein the late-onset neurodegenerative disease is amyotrophic lateral sclerosis.

Embodiment X. The acetyl-leucine for use of Embodiment VI, wherein the late-onset neurodegenerative disease is Parkinson's disease.

Embodiment XI. The acetyl-leucine for use of Embodiment VI, wherein the late-onset neurodegenerative disease is dementia.

Embodiment XII. The acetyl-leucine for use of any one of Embodiments I-III for preventing or delaying the onset of one or more symptoms of the late-onset neurodegenerative disease.

Embodiment XIII. The acetyl-leucine for use of Embodiment XII for delaying the onset of one or more symptoms of the late-onset neurodegenerative disease.

Embodiment XIV. The acetyl-leucine for use of Embodiments XII or XIII, wherein the late-onset neurodegenerative disease is Niemann-Pick type C, Alzheimer's disease, amyotrophic lateral sclerosis, Parkinson's disease, or dementia.

Embodiment XV. The acetyl-leucine for use of Embodiment XIV, wherein the late-onset neurodegenerative disease is Niemann-Pick type C.

Embodiment XVI. The acetyl-leucine for use of Embodiment XV, wherein the one or more symptoms comprise cerebellar ataxia, dysarthria, dysphagia tremor, epilepsy, vertical supranuclear palsy, sleep inversion, gelastic cataplexy, dystonia, spasticity, hypotonia, ptosis, microcephaly, psychosis, progressive dementia, progressive hearing loss, bipolar disorder, or depression.

Embodiment XVII. The acetyl-leucine for use of Embodiment XIV, wherein the late-onset neurodegenerative disease is Alzheimer's disease.

Embodiment XVIII. The acetyl-leucine for use of Embodiment XVII, wherein the one or more symptoms comprise difficulty performing familiar tasks, memory loss, disorientation to time and place, loss of good judgment, problems with abstract thinking, misplacing things, rapid mood swings, sudden and dramatic personality changes, loss of initiative, sleeping longer than usual, or loss of interest in usual activities.

Embodiment XIX. The acetyl-leucine for use of Embodiment XIV, wherein the late-onset neurodegenerative disease is amyotrophic lateral sclerosis.

Embodiment XX. The acetyl-leucine for use of Embodiment XIX, wherein the one or more symptoms comprise muscle weakness or atrophy, spasticity, trouble swallowing or breathing, cramping, or slurred and nasal speech.

Embodiment XXI. The acetyl-leucine for use of Embodiment XIV, wherein the late-onset neurodegenerative disease is Parkinson's disease.

Embodiment XXII. The acetyl-leucine for use of Embodiment XXI, wherein the one or more symptoms comprise tremor, bradykinesia, muscle stiffness, impaired posture and balance, loss of automatic movements, speech changes, or writing changes.

Embodiment XXIII. The acetyl-leucine for use of Embodiment XIV, wherein said late-onset neurodegenerative disease is dementia.

Embodiment XXIV. The acetyl-leucine for use of Embodiment XXIII, wherein the one or more symptoms comprise memory loss, difficulty communicating or finding words, difficulty with visual and spatial abilities, difficulty reasoning or problem-solving, difficulty handling complex tasks, difficulty with planning and organizing, difficulty with coordination and motor functions, confusion, or disorientation.

Embodiment XXV. The acetyl-leucine for use of any one of Embodiments I-XXIV, wherein 1 gram to 30 grams of acetyl-leucine are administered to the subject per day.

Embodiment XXVI. The acetyl-leucine for use of any one of Embodiments I-XXV, wherein the acetyl-leucine is to be administered to the subject in combination with another therapeutic agent.

Embodiment XXVII. The acetyl-leucine for use of any one of Embodiments I-XXVI wherein acetyl-DL-leucine is administered to the subject.

Embodiment XXVIII. The acetyl-leucine for use of any one of Embodiments I-XXVI wherein acetyl-L-leucine is administered to the subject.

Embodiment XXIX. The acetyl-leucine for use of any one of Embodiments I-XXVIII, wherein the NPC1 gene mutation is a c.3246-25A>G mutation.

Embodiment XXX. The acetyl-leucine for use of any one of Embodiments I-XXVIII, wherein the NPC1 gene mutation is a c.3246-5_3246-7del mutation.

Embodiment XXXI. The acetyl-leucine for use of any one of Embodiments I-XXVIII, wherein the NPC1 gene mutation is a c.2660C>T mutation.

Embodiment XXXII. The acetyl-leucine for use of any one of Embodiments I-XXVIII, wherein the NPC1 gene mutation is a c.2861C>T [S954L] mutation.

Embodiment XXXIII. The acetyl-leucine for use of any one of Embodiments I-XXVIII, wherein the NPC1 gene mutation is a c.2861C>T mutation.

Embodiment XXXIV. The acetyl-leucine for use of any one of Embodiments I-XXVIII, wherein the NPC1 gene mutation is a c.2776G>A mutation.

Embodiment XXXV. The acetyl-leucine for use of any one of Embodiments I-XXVIII, wherein the NPC1 gene mutation is a c.3010T>C mutation.

Embodiment XXXVI. The acetyl-leucine for use of any one of Embodiments I-XXVIII, wherein the NPC1 gene mutation is a c.2474A>G mutation.

Embodiment XXXVII. The acetyl-leucine for use of any one of Embodiments I-XXVIII, wherein the NPC1 gene mutation is a c.2978delG mutation.

Embodiment XXXVIII. The acetyl-leucine for use of any one of Embodiments I-XXVIII, wherein the NPC1 gene mutation is a c.3245+1dup mutation.

Embodiment XXXIX. The acetyl-leucine for use of any one of Embodiments I-XXVIII, wherein the NPC1 gene mutation is a c.1211G>A mutation.

Embodiment XL. The acetyl-leucine for use of any one of Embodiments I-XXVIII, wherein the NPC1 gene mutation is a c.1843C>T mutation.

Embodiment XLI. The acetyl-leucine for use of any one of Embodiments I-XXVIII, wherein the NPC1 gene mutation is a c.3182T>C mutation.

In another embodiment, the present disclosure provides the use of acetyl-leucine for the manufacture of a medicament for treating, preventing, or delaying the onset of a late-onset neurodegenerative disease and/or one or more symptoms thereof, wherein the subject is a heterozygous NPC1 gene mutation carrier. This is referred to as “Embodiment A-I.”

The disclosure also provides the following particular embodiments related to Embodiment A-I.

Embodiment A-II. The use of Embodiment A-I, wherein the subject has any one or more of (a) an elevated level of chitotriosidase in blood or plasma; (b) an elevated level of cholestane-3β,5α,6β-triol in blood or plasma; (c) oculomotor abnormalities; or (d) hepatosplenomegaly.

Embodiment A-III. The use of Embodiments A-I or A-II, wherein the subject is asymptomatic for the late-onset neurodegenerative disease at the time of the initial administration of the acetyl-leucine.

Embodiment A-IV. The use of any one of Embodiments A-I to A-III for preventing or delaying the onset of the late-onset neurodegenerative disease.

Embodiment A-V. The use of Embodiment A-IV for delaying the onset of the late-onset neurodegenerative disease.

Embodiment A-VI. The use of any one of Embodiments A-I to A-V, wherein the late-onset neurodegenerative disease is Niemann-Pick type C, Alzheimer's disease, amyotrophic lateral sclerosis, Parkinson's disease, or dementia.

Embodiment A-VII. The use of Embodiment A-VI, wherein the late-onset neurodegenerative disease is Niemann-Pick type C.

Embodiment A-VIII. The use of Embodiment A-VI, wherein the late-onset neurodegenerative disease is Alzheimer's disease.

Embodiment A-IX. The use of Embodiment A-VI, wherein the late-onset neurodegenerative disease is amyotrophic lateral sclerosis.

Embodiment A-X. The use of Embodiment A-VI, wherein the late-onset neurodegenerative disease is Parkinson's disease.

Embodiment A-XI. The use of Embodiment A-VI, wherein the late-onset neurodegenerative disease is dementia.

Embodiment A-XII. The use of any one of Embodiments A-I to A-III for preventing or delaying the onset one or more symptoms of the late-onset neurodegenerative disease.

Embodiment A-XIII. The use of Embodiment A-XII for delaying the onset one or more symptoms of the late-onset neurodegenerative disease.

Embodiment A-XIV. The use of Embodiments A-XII or A-XIII, wherein the late-onset neurodegenerative disease is Niemann-Pick type C, Alzheimer's disease, amyotrophic lateral sclerosis, Parkinson's disease, or dementia.

Embodiment A-XV. The use of Embodiment A-XIV, wherein the late-onset neurodegenerative disease is Niemann-Pick type C.

Embodiment A-XVI. The use of Embodiment A-XV, wherein the one or more symptoms comprise cerebellar ataxia, dysarthria, dysphagia tremor, epilepsy, vertical supranuclear palsy, sleep inversion, gelastic cataplexy, dystonia, spasticity, hypotonia, ptosis, microcephaly, psychosis, progressive dementia, progressive hearing loss, bipolar disorder, or depression.

Embodiment A-XVII. The use of Embodiment A-XIV, wherein the late-onset neurodegenerative disease is Alzheimer's disease.

Embodiment A-XVIII. The use of Embodiment A-XVII, wherein the one or more symptoms comprise difficulty performing familiar tasks, memory loss, disorientation to time and place, loss of good judgment, problems with abstract thinking, misplacing things, rapid mood swings, sudden and dramatic personality changes, loss of initiative, sleeping longer than usual, or loss of interest in usual activities.

Embodiment A-XIX. The use of Embodiment A-XIV, wherein the late-onset neurodegenerative disease is amyotrophic lateral sclerosis.

Embodiment A-XX. The use of Embodiment A-XIX, wherein the one or more symptoms comprise muscle weakness or atrophy, spasticity, trouble swallowing or breathing, cramping, or slurred and nasal speech.

Embodiment A-XXI. The use of Embodiment A-XIV, wherein the late-onset neurodegenerative disease is Parkinson's disease.

Embodiment A-XXII. The use of Embodiment A-XXI, wherein the one or more symptoms comprise tremor, bradykinesia, muscle stiffness, impaired posture and balance, loss of automatic movements, speech changes, or writing changes.

Embodiment A-XXIII. The use of Embodiment A-XIV, wherein said late-onset neurodegenerative disease is dementia.

Embodiment A-XXIV. The use of Embodiment A-XXIII, wherein the one or more symptoms comprise memory loss, difficulty communicating or finding words, difficulty with visual and spatial abilities, difficulty reasoning or problem-solving, difficulty handling complex tasks, difficulty with planning and organizing, difficulty with coordination and motor functions, confusion, or disorientation.

Embodiment A-XXV. The use of any one of Embodiments A-I to A-XXIV, wherein 1 gram to 30 grams of acetyl-leucine are administered to the subject per day.

Embodiment A-XXVI. The use of any one of Embodiments A-I to A-XXV, wherein the acetyl-leucine is to be administered to the subject in combination with another therapeutic agent.

Embodiment A-XXVII. The use of any one of Embodiments A-I to A-XXVI wherein acetyl-DL-leucine is administered to the subject.

Embodiment A-XXVIII. The use of any one of Embodiments A-I to A-XXVI wherein acetyl-L-leucine is administered to the subject.

Embodiment A-XXIX. The use of any one of Embodiments A-I to A-XXVIII, wherein the NPC1 gene mutation is a c.3246-25A>G mutation.

Embodiment A-XXX. The use of any one of Embodiments A-I to A-XXVIII, wherein the NPC1 gene mutation is a c.3246-5_3246-7del mutation.

Embodiment A-XXXI. The use of any one of Embodiments A-I to A-XXVIII, wherein the NPC1 gene mutation is a c.2660C>T mutation.

Embodiment A-XXXII. The use of any one of Embodiments A-I to A-XXVIIL wherein the NPC1 gene mutation is a c.2861C>T [S954L] mutation.

Embodiment A-XXXIII. The use of any one of Embodiments A-I to A-XXVIII, wherein the NPC1 gene mutation is a c.2861C>T mutation.

Embodiment A-XXXIV. The use of any one of Embodiments A-I to A-XXVIII, wherein the NPC1 gene mutation is a c.2776G>A mutation.

Embodiment A-XXXV. The use of any one of Embodiments A-I to A-XXVIII, wherein the NPC1 gene mutation is a c.3010T>C mutation.

Embodiment A-XXXVI. The use of any one of Embodiments A-I to A-XXVIII, wherein the NPC1 gene mutation is a c.2474A>G mutation.

Embodiment A-XXXVII. The use of any one of Embodiments A-I to A-XXVIII, wherein the NPC1 gene mutation is a c.2978delG mutation.

Embodiment A-XXXVIII. The use of any one of Embodiments A-I to A-XXVIII, wherein the NPC1 gene mutation is a c.3245+1dup mutation.

Embodiment A-XXXIX. The use of any one of Embodiments A-I to A-XXVIII, wherein the NPC1 gene mutation is a c.1211G>A mutation.

Embodiment A-XL. The use of any one of Embodiments A-I to A-XXVIII, wherein the NPC1 gene mutation is a c.1843C>T mutation.

Embodiment A-XLI. The use of any one of Embodiments A-I to A-XXVIII, wherein the NPC1 gene mutation is a c.3182T>C mutation.

In another embodiment, the present disclosure provides a pharmaceutical composition comprising acetyl-leucine for use in treating, preventing, or delaying the onset of a late-onset neurodegenerative disease and/or one or more symptoms thereof, wherein the subject is a heterozygous NPC1 gene mutation carrier. This is referred to as “Embodiment B-I.”

The disclosure provides the following particular embodiments related to Embodiment B-I.

Embodiment B-II. The pharmaceutical composition for use of Embodiment B-I, wherein the subject has any one or more of (a) an elevated level of chitotriosidase in blood or plasma; (b) an elevated level of cholestane-3β,5α,6β-triol in blood or plasma; (c) oculomotor abnormalities; or (d) hepatosplenomegaly.

Embodiment B-III. The pharmaceutical composition for use of Embodiments B-I or B-II, wherein the subject is asymptomatic for the late-onset neurodegenerative disease at the time of the initial administration of the acetyl-leucine.

Embodiment B-IV. The pharmaceutical composition for use of any one of Embodiments B-I to B-III for preventing or delaying the onset of the late-onset neurodegenerative disease.

Embodiment B-V. The pharmaceutical composition for use of Embodiment B-IV for delaying the onset of the late-onset neurodegenerative disease.

Embodiment B-VI. The pharmaceutical composition for use of any one of Embodiments B-I to B-V, wherein the late-onset neurodegenerative disease is Niemann-Pick type C, Alzheimer's disease, amyotrophic lateral sclerosis, Parkinson's disease, or dementia.

Embodiment B-VII. The pharmaceutical composition for use of Embodiment B-VI, wherein the late-onset neurodegenerative disease is Niemann-Pick type C.

Embodiment B-VIII. The pharmaceutical composition for use of Embodiment B-VI, wherein the late-onset neurodegenerative disease is Alzheimer's disease.

Embodiment B-IX. The pharmaceutical composition for use of Embodiment B-VI, wherein the late-onset neurodegenerative disease is amyotrophic lateral sclerosis.

Embodiment B-X. The pharmaceutical composition for use of Embodiment B-VI, wherein the late-onset neurodegenerative disease is Parkinson's disease.

Embodiment B-XI. The pharmaceutical composition for use of Embodiment B-VI, wherein the late-onset neurodegenerative disease is dementia.

Embodiment B-XII. The pharmaceutical composition for use of any one of Embodiments B-I to B-III for preventing or delaying the onset one or more symptoms of the late-onset neurodegenerative disease.

Embodiment B-XIII. The pharmaceutical composition for use of Embodiment B-XII for delaying the onset one or more symptoms of the late-onset neurodegenerative disease.

Embodiment B-XIV. The pharmaceutical composition for use of Embodiments B-XII or B-XIII, wherein the late-onset neurodegenerative disease is Niemann-Pick type C, Alzheimer's disease, amyotrophic lateral sclerosis, Parkinson's disease, or dementia.

Embodiment B-XV. The pharmaceutical composition for use of Embodiment B-XIV, wherein the late-onset neurodegenerative disease is Niemann-Pick type C.

Embodiment B-XVI. The pharmaceutical composition for use of Embodiment B-XV, wherein the one or more symptoms comprise cerebellar ataxia, dysarthria, dysphagia tremor, epilepsy, vertical supranuclear palsy, sleep inversion, gelastic cataplexy, dystonia, spasticity, hypotonia, ptosis, microcephaly, psychosis, progressive dementia, progressive hearing loss, bipolar disorder, or depression.

Embodiment B-XVII. The pharmaceutical composition for use of Embodiment B-XIV, wherein the late-onset neurodegenerative disease is Alzheimer's disease.

Embodiment B-XVIII. The pharmaceutical composition for use of Embodiment B-XVII, wherein the one or more symptoms comprise difficulty performing familiar tasks, memory loss, disorientation to time and place, loss of good judgment, problems with abstract thinking, misplacing things, rapid mood swings, sudden and dramatic personality changes, loss of initiative, sleeping longer than usual, or loss of interest in usual activities.

Embodiment B-XIX. The pharmaceutical composition for use of Embodiment B-XIV, wherein the late-onset neurodegenerative disease is amyotrophic lateral sclerosis.

Embodiment B-XX. The pharmaceutical composition for use of Embodiment B-XIX, wherein the one or more symptoms comprise muscle weakness or atrophy, spasticity, trouble swallowing or breathing, cramping, or slurred and nasal speech.

Embodiment B-XXI. The pharmaceutical composition for use of Embodiment B-XIV, wherein the late-onset neurodegenerative disease is Parkinson's disease.

Embodiment B-XXII. The pharmaceutical composition for use of Embodiment B-XXI, wherein the one or more symptoms comprise tremor, bradykinesia, muscle stiffness, impaired posture and balance, loss of automatic movements, speech changes, or writing changes.

Embodiment B-XXIII. The pharmaceutical composition for use of Embodiment B-XIV, wherein said late-onset neurodegenerative disease is dementia.

Embodiment B-XXIV. The pharmaceutical composition for use of Embodiment B-XXIII, wherein the one or more symptoms comprise memory loss, difficulty communicating or finding words, difficulty with visual and spatial abilities, difficulty reasoning or problem-solving, difficulty handling complex tasks, difficulty with planning and organizing, difficulty with coordination and motor functions, confusion, or disorientation.

Embodiment B-XXV. The pharmaceutical composition for use of any one of Embodiments B-I to B-XXIV, wherein 1 gram to 30 grams of acetyl-leucine are administered to the subject per day.

Embodiment B-XXVI. The pharmaceutical composition for use of any one of Embodiments B-I to B-XXV, wherein the acetyl-leucine is to be administered to the subject in combination with another therapeutic agent.

Embodiment B-XXVII. The pharmaceutical composition for use of any one of Embodiments B-I to B-XXVI, wherein acetyl-DL-leucine is administered to the subject.

Embodiment B-XXVIII. The pharmaceutical composition for use of any one of Embodiments B-I to B-XXVI, wherein acetyl-L-leucine is administered to the subject.

Embodiment B-XXIX. The pharmaceutical composition for use of any one of Embodiments B-I to B-XXVIII, wherein the NPC1 gene mutation is a c.3246-25A>G mutation.

Embodiment B-XXX. The pharmaceutical composition for use of any one of Embodiments B-I to B-XXVIII, wherein the NPC1 gene mutation is a c.3246-5_3246-7del mutation.

Embodiment B-XXXI. The pharmaceutical composition for use of any one of Embodiments B-I to B-XXVIII, wherein the NPC1 gene mutation is a c.2660C>T mutation.

Embodiment B-XXXII. The pharmaceutical composition for use of any one of Embodiments B-I to B-XXVIII, wherein the NPC1 gene mutation is a c.2861C>T [S954L] mutation.

Embodiment B-XXXIII. The pharmaceutical composition for use of any one of Embodiments B-I to B-XXVIII, wherein the NPC1 gene mutation is a c.2861C>T mutation.

Embodiment B-XXXIV. The pharmaceutical composition for use of any one of Embodiments B-I to B-XXVIII, wherein the NPC1 gene mutation is a c.2776G>A mutation.

Embodiment B-XXXV. The pharmaceutical composition for use of any one of Embodiments B-I to B-XXVIII, wherein the NPC1 gene mutation is a c.3010T>C mutation.

Embodiment B-XXXVI. The pharmaceutical composition for use of any one of Embodiments B-I to B-XXVIII, wherein the NPC1 gene mutation is a c.2474A>G mutation.

Embodiment B-XXXVII. The pharmaceutical composition for use of any one of Embodiments B-I to B-XXVIII, wherein the NPC1 gene mutation is a c.2978delG mutation.

Embodiment B-XXXVIII. The pharmaceutical composition for use of any one of Embodiments B-I to B-XXVIII, wherein the NPC1 gene mutation is a c.3245+1dup mutation.

Embodiment B-XXXIX. The pharmaceutical composition for use of any one of Embodiments B-I to B-XXVIII, wherein the NPC1 gene mutation is a c.1211G>A mutation.

Embodiment B-XL. The pharmaceutical composition for use of any one of Embodiments B-I to B-XXVIII, wherein the NPC1 gene mutation is a c.1843C>T mutation.

Embodiment B-XLI. The pharmaceutical composition for use of any one of Embodiments B-I to B-XXVIII, wherein the NPC1 gene mutation is a c.3182T>C mutation.

### Definitions

In some embodiments, the prophylactic methods described herein are contemplated for a subject, e.g., a human heterozygous NPC1 gene mutation carrier, at risk for developing a late-onset neurodegenerative disease, but who do not yet meet the clinical criteria for the diagnosis as having the disease. See, e.g., Patterson et al., Neurol Clin Pract. 7:499-511 (2017). Without being bound to a particular theory, it is believed that subjects in this preclinical disease stage represent a continuum from completely symptom-free subjects to subjects who demonstrate deficits and abnormalities but do not yet meet the clinical criteria for diagnosis as having a late-onset neurodegenerative disease. The term “asymptomatic” as used herein refers to a subject who falls within this continuum.

Methods such as Polymerase Chain Reaction (PCR) to determine whether a subject is a heterozygous NPC1 gene mutation carrier are known in the art. See, e.g., Zech et al., PLoS One 8(12):e82879 (2013).

As used herein, the singular forms “a,” “an,” and “the” include plural reference.

The term “acetyl-leucine” refers collectively to N-acetyl-DL-leucine (ADLL), or a pharmaceutically acceptable salt thereof; N-acetyl-D-leucine (ADL), or a pharmaceutically acceptable salt thereof; and N-acetyl-L-leucine (ALL), or a pharmaceutically acceptable salt thereof. The term acetyl-leucine includes isotopically-labelled analogs of N-acetyl-DL-leucine, N-acetyl-D-leucine, and N-acetyl-L-leucine, wherein one or more atoms are replaced by an atom having a different atomic mass or mass number. Examples of isotopes that can be incorporated include isotopes of hydrogen, carbon, nitrogen, and oxygen, such as ²H (or deuterium (D)), ³H, ¹¹C, ¹³C, ¹⁴C, ¹⁵N, ¹⁸O, and ¹⁷O. In one embodiment, provided is an isotopically-labelled analog of acetyl-leucine, wherein substantially all of the atoms at a position within acetyl-leucine are replaced by an atom having a different atomic mass or mass number. In another embodiment, provided is an isotopically-labelled analog of acetyl-leucine, wherein a portion of the atoms at a position within acetyl-leucine are replaced, e.g., acetyl-leucine is enriched at one or more positions with an atom having a different atomic mass or mass number. Isotopically-labelled acetyl-leucine can be prepared by methods known in the art.

In one embodiment, the N-acetyl-DL-leucine, N-acetyl-D-leucine, or N-acetyl-L-leucine used in the methods of the present disclosure is not isotopically-labelled.

In one embodiment, the isotopically-labelled analog is a deuterated analog of N-acetyl-DL-leucine, N-acetyl-D-leucine, or N-acetyl-L-leucine, wherein one or more hydrogen atoms are replaced with deuterium. In one embodiment, one hydrogen atom of N-acetyl-DL-leucine, N-acetyl-D-leucine, or N-acetyl-L-leucine is replaced with deuterium. In another embodiment, two hydrogen atoms of N-acetyl-DL-leucine, N-acetyl-D-leucine, or N-acetyl-L-leucine are replaced with deuterium. In another embodiment, three hydrogen atoms of N-acetyl-DL-leucine, N-acetyl-D-leucine, or N-acetyl-L-leucine are replaced with deuterium. In another embodiment, four hydrogen atoms of N-acetyl-DL-leucine, N-acetyl-D-leucine, or N-acetyl-L-leucine are replaced with deuterium. In another embodiment, five hydrogen atoms of N-acetyl-DL-leucine, N-acetyl-D-leucine, or N-acetyl-L-leucine are replaced with deuterium. In another embodiment, six hydrogen atoms of N-acetyl-DL-leucine, N-acetyl-D-leucine, or N-acetyl-L-leucine are replaced with deuterium.

In one embodiment, the acetyl-leucine used in the methods of the present disclosure is N-acetyl-DL-leucine, or a deuterated analog thereof. In another embodiment, the acetyl-leucine used in the methods of the present disclosure is N-acetyl-D-leucine, or a deuterated analog thereof. In another embodiment, the acetyl-leucine used in the methods of the present disclosure is N-acetyl-L-leucine, or a deuterated analog thereof. In another embodiment, the acetyl-leucine used in the methods of the present disclosure is N-acetyl-L-leucine.

The terms “administer,” “administration,” or “administering” as used herein refer to (1) providing, giving, dosing and/or prescribing by either a health practitioner or his authorized agent or under his direction, acetyl-leucine, or a pharmaceutical composition thereof, and (2) putting into, taking or consuming by the patient or person himself or herself, acetyl-leucine or a pharmaceutical composition thereof.

Any reference to “acetyl-leucine” includes pharmaceutically acceptable salts of the same, even if not expressly stated.

A “pharmaceutically acceptable salt” as referred to herein, is any salt preparation that is appropriate for use in a pharmaceutical application. Pharmaceutically acceptable salts include, but are not limited to, amine salts, such as N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chloro-benzyl-2-pyrrolidin-1′-ylmethylbenzimidazole, diethylamine and other alkylamines, piperazine, tris(hydroxymethyl)aminomethane and the like; alkali metal salts, such as lithium, potassium, sodium and the like; alkali earth metal salts, such as barium, calcium, magnesium and the like; transition metal salts, such as zinc, aluminum and the like; other metal salts, such as sodium hydrogen phosphate, disodium phosphate and the like; mineral acids, such as hydrochlorides, sulfates and the like; and salts of organic acids, such as acetates, lactates, malates, tartrates, citrates, ascorbates, succinates, butyrates, valerates, fumarates and the like.

Acetyl-leucine may be formulated and administered to a subject in accordance with known teachings in the art. For example, acetyl-DL-leucine, acetyl-D-leucine, or acetyl-L-leucine may be formulated as a pharmaceutical composition. In some embodiments, acetyl-DL-leucine, acetyl-D-leucine, or acetyl-L-leucine are administered to a subject as a part of pharmaceutical composition. Such pharmaceutical compositions comprise acetyl-DL-leucine, acetyl-D-leucine, or acetyl-L-leucine, and a pharmaceutically acceptable carrier. Reference to the pharmaceutical composition encompasses the active agent alone, i.e., acetyl-DL-leucine, acetyl-D-leucine, or acetyl-L-leucine, or in the form of a pharmaceutical composition.

The pharmaceutical composition may take any of a number of different forms depending, in particular, on the manner in which it is to be used. Thus, for example, it may be in the form of a powder, tablet, capsule, liquid, ointment, cream, gel, hydrogel, aerosol, spray, micellar solution, transdermal patch, liposome suspension or any other suitable form that may be administered to a person or animal in need of treatment.

A “pharmaceutically acceptable carrier” as referred to herein, is any known compound or combination of known compounds, e.g., excipients, carriers, etc., that are known to those skilled in the art to be useful in formulating pharmaceutical compositions. It will be appreciated that the carrier of the pharmaceutical composition should be one which is tolerated by the subject to whom it is given.

In one embodiment, the pharmaceutically acceptable carrier may be a solid, and the composition may be in the form of a powder or tablet. A solid pharmaceutically acceptable carrier may include, but is not limited to, one or more substances which may also act as flavouring agents, buffers, lubricants, stabilisers, solubilisers, suspending agents, wetting agents, emulsifiers, dyes, fillers, glidants, compression aids, inert binders, sweeteners, preservatives, dyes, coatings, or tablet-disintegrating agents. The carrier may also be an encapsulating material. In powders, the carrier may be a finely divided solid that is in admixture with the finely divided active agents according to the invention. In tablets, the active agent may be mixed with a carrier having the necessary compression properties in suitable proportions and compacted in the shape and size desired. The powders and tablets may, for example, contain up to 99% of the active agents. Suitable solid carriers include, for example, calcium phosphate, magnesium stearate, talc, sugars, lactose, dextrin, starch, gelatin, cellulose, polyvinylpyrrolidine, low melting waxes and ion exchange resins. In another embodiment, the pharmaceutically acceptable carrier may be a gel and the composition may be in the form of a cream or the like.

The carrier may include, but is not limited to, one or more excipients or diluents. Examples of such excipients are gelatin, gum arabicum, lactose, microcrystalline cellulose, starch, sodium starch glycolate, calcium hydrogen phosphate, magnesium stearate, talcum, colloidal silicon dioxide, and the like.

In another embodiment, the pharmaceutically acceptable carrier may be a liquid. In one embodiment, the pharmaceutical composition is in the form of a solution. Liquid carriers are used in preparing solutions, suspensions, emulsions, syrups, elixirs and pressurized compositions. Acetyl-leucine may be dissolved or suspended in a pharmaceutically acceptable liquid carrier such as water, an organic solvent, a mixture of both or pharmaceutically acceptable oils or fats. The liquid carrier may contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid carriers for oral and parenteral administration include water (partially containing additives as above, e.g. cellulose derivatives, such as sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier may also be an oily ester such as ethyl oleate and isopropyl myristate. Sterile liquid carriers are useful in sterile liquid form compositions for parenteral administration. The liquid carrier for pressurised compositions may be a halogenated hydrocarbon or other pharmaceutically acceptable propellant.

Liquid pharmaceutical compositions, which are sterile solutions or suspensions, may be utilised by, for example, intramuscular, intrathecal, epidural, intraperitoneal, intravenous and subcutaneous injection. The active agent may be prepared as a sterile solid composition that may be dissolved or suspended at the time of administration using sterile water, saline, or other appropriate sterile injectable medium.

The compositions may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The compositions may also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

Compositions may alternatively be administered by inhalation (e.g. intranasally). Compositions may also be formulated for topical use. For instance, creams or ointments may be applied to the skin.

Acetyl-leucine may be incorporated within a slow- or delayed-release device. Such devices may, for example, be inserted on or under the skin, and the medicament may be released over weeks or even months. Such devices may be advantageous when long-term treatment with acetyl-leucine used according to the present disclosure is required and which may require frequent administration (e.g. at least daily administration).

In one embodiment, the pharmaceutical composition is a solid oral dosage form, such as a tablet. In tablets, the active agent may be mixed with a vehicle, such as a pharmaceutically acceptable carrier, having the necessary compression properties in suitable proportions and compacted in the shape and size desired. The tablets may contain up to 99% by weight of the active agents.

Pharmaceutical compositions in solid oral dosage form, such as tablets, may be prepared by any method known in the art of pharmacy. Pharmaceutical compositions are usually prepared by mixing the active agent with conventional pharmaceutically acceptable carriers.

A tablet may be formulated as is known in the art. Tanganil®, for example, includes wheat starch, pregelatinised maize (corn) starch, calcium carbonate and magnesium stearate as excipients. The same, or similar, excipients, for example, may be employed with the present disclosure.

The composition of each 700 mg Tanganil® tablet is as follows: 500 mg acetyl-DL-leucine, 88 mg wheat starch, 88 mg pregelatinised maize (corn) starch, 13 mg calcium carbonate and 11 mg magnesium stearate. The same tablets, for example, may be employed in the methods of the present disclosure.

As discussed above, acetyl-leucine may be formulated and administered as a pharmaceutical composition taking any number of different forms. For example, acetyl-leucine may be formulated as a pharmaceutical composition to facilitate its delivery across the blood-brain barrier. As a further example, acetyl-leucine may be formulated as a pharmaceutical composition for bypassing the blood-brain barrier. Formulations that facilitate delivery across the blood-brain barrier or that are suitable for administration in a manner that bypasses the blood-brain barrier may be used to prepare and administer leucine (not acetylated) as described herein.

In one embodiment, the pharmaceutical composition, e.g., comprising acetyl-L-leucine, or salt thereof, is formulated for nanodelivery, e.g., colloidal drug-carrier systems. Suitable examples include but are not limited to liposomes, nanoparticles (e.g., polymeric, lipid and inorganic nanoparticles), nanogels, dendrimers, micelles, nanoemulsions, polymersomes, exosomes, and quantum dots. See, e.g., Patel et al., “Crossing the Blood-Brain Barrier: Recent Advances in Drug Delivery to the Brain,” CNS Drugs 31:109-133 (2017); Kabanov et al., “New Technologies for Drug Delivery across the Blood Brain Barrier,” Curr Pharm Des., 10(12):1355-1363 (2004); Cheng et al., “Highly Stabilized Curcumin Nanoparticles Tested in an In Vitro Blood-Brain Barrier Model and in Alzheimer's Disease Tg2576 Mice,” The AAPS Journal, vol. 15, no. 2, pp. 324-336 (2013); Lifhde et al. “Production of L-Leucine Nanoparticles under Various Conditions Using an Aerosol Flow Reactor Method,” Journal of Nanomaterials, vol. 2008, article ID 680897 (2008).

In one embodiment, the pharmaceutical composition, e.g., comprising acetyl-L-leucine, or salt thereof, is formulated for direct delivery to the central nervous system (CNS), such as by injection or infusion. Formulations for and methods of direct delivery to the CNS are known in the art. See, e.g., U.S. Pat. No. 9,283,181. Examples of such administration include but are not limited to intranasal, intraventricular, intrathecal, intracranial, and delivery via nasal mucosal grafting.

In one embodiment, the pharmaceutical composition is formulated for (and administered by) intranasal delivery. See, e.g., Hanson et al., “Intranasal delivery bypasses the blood-brain barrier to target therapeutic agents to the central nervous system and treat neurodegenerative disease,” BMC Neurosci. 9(Suppl 3):S5 (2008). In one embodiment, the pharmaceutical composition is formulated for (and administered by) delivery via a nasal mucosal graft. In one embodiment, the pharmaceutical composition is formulated for (and administered by) intracerebroventricular injection or infusion. In another embodiment, the pharmaceutical composition is formulated for (and administered by) intrathecal intracisternal injection or infusion. In one embodiment, the pharmaceutical composition is formulated for (and administered by) intrathecal lumbar injection or infusion.

Various techniques may be used including, without limitation, injection through a burrhole or cisternal or lumbar puncture or the like as known in the art. Various devices, whether internal (e.g., implanted) or external, may be used for delivery as known in the art, such as pumps, catheters, reservoirs, etc. In one embodiment, the administration interval is once every two weeks.

In one embodiment, the administration interval is once every month. In one embodiment, the administration interval is once every two months. In one embodiment, the administration interval is twice per month. In one embodiment, the administration interval is once every week. In one embodiment, the administration interval is twice or several times per week. In one embodiment, the administration interval is daily. In one embodiment, the administration is continuous, such as continuous infusion.

In one embodiment, the dose or amount equivalent of acetyl-leucine may adjusted to account for either its direct delivery to the CNS or its delivery across the blood-brain barrier.

A “therapeutically effective amount” of acetyl-leucine is any amount which, when administered to a subject, is the amount that is needed to produce the desired effect, which, for the present disclosure, can be therapeutic and/or prophylactic. The dose may be determined according to various parameters, such as the age, weight and condition of the patient to be treated; the route of administration; and the required regimen. A physician will be able to determine the required route of administration and dosage for any particular patient. For example, a daily dose may be from about 10 to about 225 mg per kg, from about 10 to about 150 mg per kg, or from about 10 to about 100 mg per kg of body weight.

As used herein, “treating” or “treatment” refers to any indicia of success in preventing, delaying the onset of, arresting, or ameliorating a disease, disorder, condition, or syndrome, e.g., late-onset neurodegenerative disease, in a subject, including any objective or subjective parameter such as abatement; remission; preventing, diminishing, inhibiting, or eliminating one or more symptoms; making the disease, disorder, condition, or syndrome more tolerable to the subject; slowing in the worsening of the disease, disorder, condition, or syndrome; or improving the physical or mental well-being of the subject in need thereof.

The terms “treating” or “treatment” also encompasses, e.g., inducing inhibition, regression, or stasis of the disease, disorder, condition, or syndrome. For example, treatment of a subject in need of treatment for a late-onset neurodegenerative disease includes reducing a symptom of the late-onset neurodegenerative disease in the subject, inducing clinical response, inhibiting or reducing progression of the late-onset neurodegenerative disease, or inhibiting or reducing a complications of the late-onset neurodegenerative disease.

Preventing, arresting, or ameliorating an injury or pathology of a disease, disorder, condition, or syndrome, such as preventing, diminishing, inhibiting, or eliminating one or more symptoms of disease, disorder, condition, or syndrome can be based on objective and/or subjective parameters, including, e.g., the results of physical examination(s), neurological examination(s), and/or psychiatric evaluation(s). The success of treatment for certain late-onset neurodegenerative diseases may be measured or evaluated by, for example, comparing the severity of the disease (e.g., objective and/or subjective parameters of NPC) before treatment with acetyl-leucine is initiated, with the severity of the disease (e.g., objective and/or subjective parameters of the late-onset neurodegenerative disease) following the initiation of treatment with acetyl-leucine. For example, the severity of the late-onset neurodegenerative disease may be assessed using a scale, index, rating, or score. In one embodiment, the treatment described herein improves such an assessment from a value or degree characteristic of a symptomatic subject to a value or degree characteristic of a non-symptomatic subject. In one embodiment, the treatment described herein improves such an assessment compared to a baseline. The baseline may be, for example, the subject's condition before initiating any treatment for the disease or before initiating treatment for the disease with acetyl-leucine. Alternatively, the baseline may be, for example, the subject's condition after a certain time period on treatment for the disease. In one embodiment, treatment with acetyl-leucine as described herein improves the subject's assessment (e.g., scale, index, rating, or score of objective and/or subjective parameters) compared to a baseline by at least 5%, at least 10%, at least 20%, at least 30%, at least 40%, or at least 50%. In one embodiment, assessment is improved by at least 60%, at least 70%, at least 80%, at least 90%, or 100%.

In one embodiment, the severity of the subject's late-onset neurodegenerative disease or symptom thereof may be quantified by neurological and/or neuropsychological assessments such as abdominal ultrasound with volumometry, video-oculography (VOG), and/or the testing battery proposed by the Consortium to Establish a Registry for Alzheimer's Disease (CERAD).

A “symptom” of a late-onset neurodegenerative disease, includes any clinical or laboratory manifestation associated with the late-onset neurodegenerative diseases, and is not limited to what the subject can feel or observe. The symptoms of late-onset neurodegenerative diseases are known in the art.

Symptoms of NPC include, but are not limited to, cerebellar ataxia (unsteady walking with uncoordinated limb movements), dysarthria (slurred speech), dysphagia (difficulty in swallowing), tremor, epilepsy, vertical supranuclear palsy (upgaze palsy, downgaze palsy, saccadic palsy or paralysis), sleep inversion, gelastic cataplexy (sudden loss of muscle tone or drop attacks), dystonia (abnormal movements or postures caused by contraction of agonist and antagonist muscles across joints), spasticity (velocity dependent increase in muscle tone), hypotonia, ptosis (drooping of the upper eyelid), microcephaly (abnormally small head), psychosis, progressive dementia, progressive hearing loss, bipolar disorder, major and psychotic depression that can include hallucinations, delusions, mutism, or stupor.

Symptoms of AD include, but are not limited to, difficulty performing familiar tasks, memory loss, disorientation to time and place, loss of good judgment, problems with abstract thinking, misplacing things, rapid mood swings, sudden and dramatic personality changes, loss of initiative, sleeping longer than usual, or loss of interest in usual activities

Symptoms of amyotrophic lateral sclerosis (ALS) include, but are not limited to, muscle weakness or atrophy, spasticity, trouble swallowing or breathing, cramping, or stiffness of affected muscles; or slurred and nasal speech.

Symptoms of Parkinson's disease include, but are not limited to, tremor, bradykinesia, muscle stiffness, impaired posture and balance, loss of automatic movements, speech changes, or writing changes.

Symptoms of dementia include, but are not limited to, memory loss, which is usually noticed by a spouse or someone else, difficulty communicating or finding words, difficulty with visual and spatial abilities, such as getting lost while driving, difficulty reasoning or problem-solving, difficulty handling complex tasks, difficulty with planning and organizing, difficulty with coordination and motor functions, confusion, or disorientation.

In one embodiment, acetyl-leucine may be administered, for example, at a dose ranging from about 500 mg to about 30 g per day or ranging from about 500 mg to about 15 g per day, such as ranging from about 1.5 g to about 10 g per day, optionally by solid oral or liquid oral route. Acetyl-leucine, may be administered, for example, in a dose according to that of Tanganil®, which is prescribed to adults in a dose of 1.5 g to 2 g per day, 3-4 tablets in two doses, morning and evening.

If a single enantiomer, i.e., acetyl-L-leucine, is administered the doses may be reduced accordingly. For instance, if only acetyl-L-leucine is administered, the dose may range from about 250 mg to about 15 g per day, range from about 250 mg to about 10 g per day, or range from about 250 mg to about 5 g per day, such as from about 0.75 g to about 5 g per day.

In one embodiment, the administered dose ranges from about 1 g to about 30 g per day, from about 1 g to about 15 g per day, from about 1 g to about 10 g per day, or from about 1.5 g to about 7 g per day, from 15.1 g to about 30 g per day, 16 g to about 30 g per day, 17 g to about 30 g per day, 18 g to about 30 g per day, 19 g to about 30 g per day, or 20 g to about 30 g per day. It may be from about 2, 3, 4, 5, 6, 7, 8, 9, 10, 11, 12, 13, or 14 g to about 15 g per day. It may be from about 2, 3, 4, 5, 6, 7, 8 or 9 g to about 10 g per day. It may be from 15.1, 16, 17, 18, 19, 20, 21, 22, 23, 24, 25, 25, 27, 28, or 29 g to about 30 g per day. It may be more than about 1.5 g per day, but less than about 15, 14, 13, 12, 11, 10, 9, 8, 7, 6 or 5 g per day. In one embodiment, the dose ranges from about 4 g to about 6 g per day. In one embodiment, the dose ranges from about 4 g to about 5 g per day. In one embodiment, the dose is about 4.5 g per day. In one embodiment, the dose is about 5 g per day. In one embodiment, the dose is about 1 g per day, about 2 g per day, about 3 g per day, about 4 g per day, about 5 g per day, about 6 g per day, about 7 g per day, about 8 g per day, about 9 g per day, about 10 g per day, about 11 g per day, about 12 g per day, about 13 g per day, about 14 g per day, or about 15 g per day. In another embodiment, the dose is about 16 g per day, about 17 g per day, about 18 g per day, about 19 g per day, or about 20 g per day. In another embodiment, the dose is about 21 g per day, about 22 g per day, about 23 g per day, about 24 g per day, about 25 g per day, about 26 g per day, about 27 g per day, about 28 g per day, about 29 g per day, or about 30 g per day. In one embodiment, these doses are administered in a solid oral dosage form, notably tablets. In another embodiment, these doses are for acetyl-leucine when in its racemic form. Doses for acetyl-leucine when an enantiomeric excess is present may be lower, for example, around 50% lower. The above recited dose-ranges when halved are thus also explicitly encompassed by the present disclosure.

Also, in some embodiments, asymptomatic subjects may receive lower doses of acetyl-leucine, e.g., 1 g to 10 g per day, as compound to subjects that have been clinically diagnosed as having a late-onset neurodegenerative disease who may receive, e.g., 5 g to 15 g of acetyl-leucine per day.

In one embodiment, the total daily dose may be spread across multiple administrations, i.e. administration may occur two or more times a day to achieve the total daily dose. As an example, the required number of tablets to provide the total daily dose of acetyl-leucine may be split across two administrations (for example, in the morning and evening) or three administrations (for example, in the morning, noon, and evening). Each dose may be suitably administered with or without food. For example, acetyl-L-leucine or acetyl-DL-leucine may be dosed by about 1 or about 2 hours before meals, such as at least about 20 minutes, at least about 30 minutes, at least about 40 minutes, or at least about 1 hour before meals, or may be dosed by about 1, about 2, or about 3 hours after meals, such as waiting at least about 20 minutes, at least about 30 minutes, at least about 1 hour, at least about 1.5 hours, at least about 2 hours, or at least about 2.5 hours after meals. For example, a total daily dose of 4.5 g acetyl-DL-leucine may be administered as three Tanganil® (or equivalent) tablets before, with, or after breakfast, three further tablets before, with, or after lunch and three further tablets before, with, or after dinner.

Treatment duration may be, for example, about seven days or more, about two weeks or more, about three weeks or more, about one month or more, about six weeks or more, about seven weeks or more, or about two months or more. In one embodiment, it is about three months or more, about four months or more, about five months or more or about six months or more. The treatment duration may be about 1 year or more, about 2 years or more, about 4 years or more, about 5 years or more, or about 10 years or more. The treatment duration may be the life-time of the subject.

Any and all combinations of dosage form, dose amount, dosing schedule and treatment duration are envisaged and encompassed by the disclosure. In one embodiment, the dose is from about 4 g to about 10 g per day, taken across one, two, or three administrations per day, for a treatment duration of about two months or more. In another embodiment, the dose is more than 4 g but no more than 5 g per day, taken across one, two, or three administrations per day, for a treatment duration of about six months or more. The dosage form may be a solid oral dosage form, notably tablets.

Acetyl-leucine may be used as a monotherapy, e.g., used as the active agent alone, for treating or preventing the late-onset neurodegenerative disease, or the symptoms thereof, in a subject. Alternatively, acetyl-leucine may be used as an adjunct to, or in combination with, other known therapies for treating or preventing the late-onset neurodegenerative disease.

Also disclosed is a kit for treating, preventing, or delaying the onset of a late-onset neurodegenerative disease, comprising acetyl-leucine. The kit may further comprise instructions for using acetyl-leucine according to a method of the present disclosure and/or a means for diagnosing or prognosing the disease.

### EXAMPLES

### General Study Parameters

Twenty first-degree relatives of genetically and/or biochemically confirmed NPC patients participated (13 males, mean age 52.7±9.9, education years 14.2±2.4) were recruited between 2016 and 2018. All probands were molecularly-confirmed heterozygous NPC1 gene mutation carriers for this study. Participants were of German, Turkish, Slovakian, Spanish, and Swedish descent. None of the participants took neuropotent medication. The following battery was chosen to recapitulate characteristic features of symptomatic NPC disease and to screen for abnormalities suggestive of late-onset neurodegeneration. All underwent neurological evaluation with particular focus on movement abnormalities. A motor score was defined giving 1 point each for presence of a reduced arm swing, intention tremor, increased muscle tone, ankle clonus, gait abnormalities. Scores ≥1 were considered pathological. Grooved PEG-board testing to assess fine motor function was performed as previously described (Strauss 2006). It is routinely administered in patients with lysosomal storage disorders (Bremova-Ertl et al. 2017).

Mood was evaluated using the Beck depression scale (Bech et al. 1961); REM-sleep behaviour was assessed using the REM-sleep Behaviour Disorder (RBD) questionnaire (Stiasny-Kolster et al. 2014). Scented Sniffin' Sticks pens were used to assess smell identification, threshold and discrimination (Hummel et al. 1997).

In seventeen subjects, standardized neuropsychological assessment was performed in their native language using the battery proposed by the Consortium to Establish a Registry for Alzheimer's Disease (CERAD) (Ehrensperger et al. 2010). The remaining three were too young for the CERAD analysis. In two individuals, a subset of tests could not be performed because of language incongruences (see Tables 2A and 2B). CERAD includes assessment of global cognitive function (mini mental state examination, [MMSE]), executive functions, e.g., trail making test (Arbuthnott et al. 2000; Sanchez-Cubillo et al. 2009)) to examine mental processing speed [TMT-A]and set-shifting [TMT-B], TMTB-A, which is a relatively pure indicator of executive control, lexical and semantic fluency), attention (verbal and visual span), memory (including the three components of encoding, storage and retrieval, i.e. word list direct recall [directCERAD], word list delayed recall [delayedCERAD], word list recognition [savings]), language (Boston naming test, [BNT]), and visuo-constructive functions. Constructional praxis (CP) copying task consists of four figures (circle, diamond, overlapping rectangles, Necker cube) was also assessed. The CLOX test, an executive clock drawing task, was also administered (Royall et al. 1998). Individual raw scores were transformed to z-scores based on a large database of healthy control subjects considering, age, education, and gender (Luppa et al. 2012).

Given the characteristic ocular motor deficits in NPC, i.e. vertical supranuclear gaze palsy, VOG was performed in 16 heterozygotes using a video-based eye-tracker system (EyeSeeCam®, Munich, Germany) as previously described (Schneider et al. 2009). The data was compared to 36 age- and sex-matched controls. Briefly, the following parameters were assessed: reflexive saccades which reflect brainstem function, self-paced saccades (an ocular motor task with a cognitive dimension to assess the frontal eye field), smooth pursuit, gaze-holding, and horizontal vestibulo-ocular reflex.

Blood was analysed for NPC biomarkers, i.e. plasma chitotriosidase activity and concentration of cholestane-3β,5α,6β-triol as previously described (Reunert et al 2015). Selected routine parameters including full blood count and routine liver function tests were also measured.

Abdominal ultrasound with volumometry was performed (n=12) to assess the size of liver and spleen, as used in routine clinical practice.

¹⁸FDG-PET (n=16) was performed using a Siemens Biograph 64 PET/CT scanner as previously described (Beyer et al. 2018; Brendel et al. 2017); visual judgment of significant hypometabolism in different brain areas was based on stereotactic surface projections and axial slices; a binarized decision of significant or non-significant hypometabolism was performed in clinical routine; for quantitative analyses PMOD Version 3.5 (PMOD Technologies Ltd., Zurich, Switzerland) was used for spatial normalization of all images to the Montreal Neurological Institute (MNI) space and for activity normalization by the global mean. Statistical parametric mapping (SPM) was performed using SPM12 implemented in Matlab (R2016). Subjects were compared against 23 age-matched in-house healthy controls (departmental data available) by a voxel-wise two-tailed Student's t-test after 8 mm Gaussian smoothing. The significance threshold was set to p<0.01 (uncorrected for multiple comparisons; k>20 voxel). Based on resulting significant clusters, uptake values from ten volumes-of-interest were extracted. They were as follows: left anterior cingulate cortex and bilateral posterior cingulate cortex, bilateral cerebellum, basal ganglia, parietal cortex left, bilateral temporal cortex, bilateral insular cortex, bilateral midbrain and tegmentum, and bilateral postcentral cortex.

### Statistical Analysis

Statistical analysis and graphical design were performed using SPSS version 25.0.0 (IBM, Armonk, NY). Normality of data distribution was tested using the mean, median, SD, skewness, kurtosis, and box plots. For data that were normally distributed, t-test for related and non-related variables, respectively, were used (¹⁸FDG-PET outcomes). Data that were not normally distributed, nonparametric paired Wilcoxon test for related groups and non-paired testing using the Mann-Whitney-U test in case of non-related samples, were performed (VOG measures). Differences were considered significant if p<0.05. Spearmen's Correlation Coefficient was calculated to assess the relationships between clinical data and scores, neuropsychological scores and imaging data.

More than 80% of assessments were completed by all 20 participants.

Demographic characteristics and key findings are summarized in Tables 1A-1C.

TABLE 1A

| Subject | | | Country | | Motor |
|---|---|---|---|---|---|
| ID | Sex | Age | of origin | Genotype (NPC1) | score ^{a} |
| 1 | M | 68 | GER | c.2660C > T | 2 |
| 2 | M* | 64 | GER | c.2861C > T [S954L] | 2 |
| 3 | F* | 55 | GER | c.2861C > T [S954L] | 0 |
| 4 | F\hat{ } | 53 | GER | c 3246-5_3246-7del^{x} | 0 |
| 5 | M\hat{ } | 61 | GER | c 3246-5_3246-7del^{x} | 5 |
| 6 | M | 51 | TUR | c.3246-25A > G^{xx} | 1 |
| 7 | M° | 61 | GER | c.2861C > T | 0 |
| 8 | F° | 59 | GER | c.2776G > A | 1 |
| 9 | F | 54 | GER | c.2861C > T | 1 |
| 10 | M* | 25 | SLOV | c.2861C > T | 0 |
| 11 | M* | 62 | SLOV | c.2861C > T | 2 |
| 12 | F | 46 | GER | c.3010T > C | 1 |
| 13 | M | 55 | GER | c.2474A > G | 0 |
| 14 | M | 48 | GER | c.2474A < G | 1 |
| 15 | M\hat{ } | 39 | ESP | c.2978delG, | 2 |
| 16 | F\hat{ } | 39 | ESP | c.3245 + 1dup | 0 |
| 17 | F° | 49 | SWED | c.1211G > A | 0 |
| 18 | M° | 50 | SWED | c.1843C > T | 2 |
| 19 | M* | 57 | GER | c.3182T > C | 1 |
| 20 | F* | 57 | GER | c.3182T > C | 1 |

| | |
|---|---|
| Abnormal (%) | 65% |

TABLE 1B

| | Grooved-PEG | | | Smell |
|---|---|---|---|---|
| Subject ID | board (Dominant) | Depression ^{b} | RBDSQ ^{c} | test ^{d} |
| 1 | 11 | 10 | 1 | 13 |
| 2 | 9 | 0 | 1 | 13 |
| 3 | 14 | 22 | 6 | 13 |
| 4 | 15 | 14 | 9 | 12 |
| 5 | 8 | 5 | 4 | 13 |
| 6 | 13 | n.d. | 0 | 13 |
| 7 | 9 | 0 | 2 | 10 |
| 8 | 16 | 5 | 4 | 13 |
| 9 | 14 | 19 | 1 | 8 |
| 10 | 14 | n.d. | n.d. | 11 |
| 11 | 16 | n.d. | n.d. | 13 |
| 12 | 16 | 9 | 1 | 14 |
| 13 | 12 | 11 | 6 | 11 |
| 14 | 13 | 11 | 1 | 13 |
| 15 | 16 | 7 | 5 | 13 |
| 16 | 18 | n.d. | n.d. | 14 |
| 17 | 17 | 3 | 0 | 13 |
| 18 | 15 | 0 | 1 | 13 |
| 19 | 17 | 7 | 4 | 13 |
| 20 | 11 | 8 | 4 | 14 |
| Abnormal (%) | | 37.5% | 24% | 20% |

TABLE 1C

| | | | | | FDG |
|---|---|---|---|---|---|
| | | | Chitotriosidase | | PET^{g} |
| | Abdominal | | activity | Oxysterols | (visual |
| Subject | ultrasound ^{e} | | [nmol/h/ml] | [ng/μl] | rating |
| ID | Spleen | Liver | (norm ≤150) | (norm ≤0.05) ^{f} | 0-12) |
| 1 | − | + | 699.4 | 0.037 | 6 |
| 2 | − | ++ | 103.4 | 0.026 | 4 |
| 3 | − | − | 39.0 | 0.027 | 3 |
| 4 | − | + | 74.7 | 0.022 | 3 |
| 5 | − | + | 80.7 | 0.012 | 2 |
| 6 | n.d. | n.d. | 10.1 | 0.014 | n.d. |
| 7 | + | − | 164.5 | 0.064 | 6 |
| 8 | − | + | 33.4 | 0.063 | 1 |
| 9 | n.d. | n.d. | 45.2 | 0.02 | 5 |
| 10 | n.d. | n.d. | n.d. | n.d. | 0 |
| 11 | n.d. | n.d. | n.d. | n.d. | 2 |
| 12 | n.d. | n.d. | 35.4 | 0.021 | n.d. |
| 13 | ++ | ++ | 171.9 | 0.187 | 1 |
| 14 | ++ | − | 113.7 | 0.048 | 1 |
| 15 | ++ | − | 20.8 | 0.052 | 0 |
| 16 | − | − | 85.6 | 0.028 | n.d. |
| 17 | − | − | 28.8 | 0.021 | 2 |
| 18 | − | ++ | 57.7 | 0.037 | 1 |
| 19 | n.d. | n.d. | 51.6 | 0.054 | 4 |
| 20 | − | − | 77.8 | 0.075 | n.d. |

| | | | | |
|---|---|---|---|---|
| Abnormal | 71% | 17% | 33% | 50% |
| (%) | | | | |

Abbreviations for Tables 1A-1C: n.d.=no data. N. y. f.=not yet found. Country of origin: GER—Germany, TUR—Turkey, SLOV—Slovakia, ESP—Spain, SWED—Sweden. The symbols *, ∘, \hat{ } indicate participants from the same family. ^{X} An intronic variant not previously described in the literature (class 3), leading to skipping of exon 22. ^{xx} This intronic mutation has not been described yet. ENST00000269228, position in the genome: chr18:21115689. a=A motor score was defined giving 1 point each for presence of a reduced arm swing, intention tremor, increased muscle tone, ankle clonus, gait abnormalities. Scores ≥1 were considered pathological; b=using the Beck Depression Inventory with 0-9 indicating that a person is not depressed, 10-18 indicating mild-moderate depression, 19-29 indicating moderate-severe depression and 30-63 indicating severe depression; c=REM sleep behavior was assessed using the validated a 10-item patient self-rating questionnaire (maximum total score 13 points) covering the clinical features of RBD; values ≥5 are considered pathological; d=Odor identification was performed using Sniffin's sticks; results <12 are considered pathological. Apart from subject 11, all were non-smokers; e=Ultrasound findings were scored as follows: − for normal findings, + for mild enlargement of liver and/or spleen, ++ for definite organomegaly of liver and/or spleen; f=Oxysterols (cholestane-3β,5α,6β-triol); g=¹⁸FDG-PET was rated as 1, 2, or 3 when discrete, moderate or definite abnormalities were present in the parietal cortex, the temporal cortex, the anterior cingulate cortex and the cerebellum; normal PET findings were rated as “0”. The resulting subscores of all regions were summed to a combined FDG-PET score.

### Example 1

### Clinical Findings

Motor abnormalities were present in 13 of 20 participants (65%), as detected on clinical examination or PEG-board testing. Six subjects (of 16; 37.5%) reported symptoms of mild or moderate depression (BDI≥210), screening for REM sleep behaviour disorder was positive in almost one quarter (4 of 17; 24%); four participants (20%) were hyposmic. Chitotriosidase activity and cholestantriol levels were abnormal in three and six subjects (17%; 33%), respectively. Transaminase levels (GPT, GOT and GGT) were elevated in one subject (subject #11).

### Example 2

### Cognitive Testing

The results of neuropsychological testing by CERAD subdomain are presented in Tables 2A and 2B and FIG. 1. Twelve participants completed the whole CERAD battery. Global cognitive performance was impaired in three subjects who scored 26 or less on the MMSE (mean MMSE across all participants 27.87±2.59 with a minimum of 21 points in subject #6; mean age-dependent z-score −1.38±1.69 with a minimum of −4.98 in subject #6). Word list retrieval was ≤80% in eight heterozygotes (minimum, 58% in subject #6). Word list recognition discriminability was overall preserved with only mild abnormalities in three subjects. Word list savings varied between 58% and 113% with a mean of 90.6±15% (percentage >100% reflects better performance than in the sex-/age-matched normative sample). Mean percental savings of characters was 86.3±20% (range, 44-111%). The phonematic fluency reached a mean of 12.4±5.4 words, Boston Naming Test (BNT) yielded 14.4±1.8 words, and an average of 22.7±6.4 animals were named in the verbal fluency test. Mean TMT A was 30.76±10.84 s; mean TMT B was 68.14±24.22 s. TMT B-A time (calculated as a subtraction of TMT-A (numbers) and TMT-B (number and letters)) varied remarkably between subjects, yielding a mean of 37.3821.29 s with a maximum of 93.10 s. Four subjects scored in the pathological range when related to an age-matched normative sample (z-value ≤−1). The CLOX test was abnormal in five participants (score ≤9).

TABLE 2A

| Subject | | | Education | | Word | Word list | Savings | Figure |
|---|---|---|---|---|---|---|---|---|
| ID | Sex | Age | (yrs) | MMSE | list total | recall | %^{a} | drawing |
| Maximum score | | | | 30 | | 10 | 100 | 11 |
| 1 | M | 68 | 11 | 25 | 16 | 5 | 84 | 9 |
| 2 | M | 64 | 17 | 27 | 19 | 7 | 100 | 8 |
| 3 | F | 55 | 13 | 24 | 20 | 9 | 113 | 9 |
| 4 | F | 53 | 13 | 30 | 23 | 8 | 80 | 9 |
| 5 | M | 61 | 16 | 29 | 23 | 6 | 75 | 9 |
| 6 | M | 51 | 9 | 21 | 22 | 7 | 88 | 8 |
| 7 | M | 61 | 13 | 29 | 18 | 4 | 58 | 10 |
| 8 | F | 59 | 14 | 29 | 18 | 7 | 88 | 9 |
| 9 | F | 54 | 12 | 29 | 24 | 8 | 80 | 7 |
| 12^{$} | F | 46 | 15 | 29 | 29 | 10 | 100 | 10 |
| 13 | M | 55 | 17 | 30 | 21 | 9 | 113 | 10 |
| 14^{$} | M | 48 | 13 | 28 | 19 | 7 | 100 | 10 |
| 15^{$} | M | 39 | 17 | 30 | 30 | 10 | 100 | 11 |
| 17^{$$} | F | 49 | 17 | n/a | n/a | n/a | n/a | n/a |
| 18^{$$} | M | 50 | 17 | n/a | n/a | n/a | n/a | n/a |
| 19 | F | 57 | 13 | 29 | 21 | 9 | 100 | 9 |
| 20 | M | 57 | 14 | 29 | 20 | 8 | 80 | 8 |

TABLE 2B

| | Savings % | | Executive | | | | |
|---|---|---|---|---|---|---|---|
| Subject | of | Temporal | function | TMT A | TMT B | TMT B-A | |
| ID | characters^{a} | Sum^{b} | Sum^{c} | [sec] | [sec] | [sec] | CLOX^{d} |
| Maximum | 100 | 5 | 5 | | | | 15 |
| score | | | | | | | |
| 1 | 100 | 1 | 3 | 48.9 | 119.2 | 70.3 | 15 |
| 2 | 88 | 0 | 2 | 43 | 85 | 42 | 8 |
| 3 | 89 | 1 | 0 | 22.3 | 58.2 | 35.9 | 10 |
| 4 | 111 | 1 | 0 | 36.1 | 48.3 | 12.2 | 7 |
| 5 | 67 | 2 | 1 | 41.5 | 52.2 | 10.7 | 9 |
| 6 | 88 | 0 | 3 | 25 | 118.1 | 93.1 | 11 |
| 7 | 60 | 3 | 3 | 28.4 | 87.6 | 59.2 | 13 |
| 8 | 44 | 2 | 1 | 44.5 | 80.4 | 35.9 | 12 |
| 9 | 129 | 1 | 0 | 25.7 | 44.8 | 19.1 | 9 |
| 12^{$} | 80 | n/a | 0 | 18 | 54.7 | 36.7 | 13 |
| 13 | 70 | 1 | 0 | 27.6 | 65.1 | 37.5 | 11 |
| 14^{$} | 90 | n/a | 1 | 24.5 | 63.2 | 38.7 | 9 |
| 15^{$} | 82 | n/a | 0 | 15.3 | 32.6 | 17.3 | 11 |
| 17^{$$} | n/a | n/a | 0 | 13.4 | 46 | 32.6 | 12 |
| 18^{$$} | n/a | n/a | 2 | 32.7 | 79 | 46.3 | 10 |
| 19 | 100 | 1 | 1 | 44 | 65 | 21 | 13 |
| 20 | 100 | 0 | 1 | 32 | 59 | 27 | 13 |

Abbreviations for Tables 2A and 2B: $=patients were too young for formal CERAD analysis. z-scores could thus not be calculated. $$=patients were non-fluent in German, thus language-dependent aspects of cognitive testing were not performed. z-scores could thus not be calculated. Probands 10, 11 and 16 did not participate in the CERAD testing. a=Subjects with >100%/performed better in the delayed than the direct recall; b=Sum scores were calculated based on total word list, word list recall, word list savings %, word list discriminability, figure savings %. Scores >2 were considered pathological; c=Sum scores were calculated based on phonetic and semantic verbal fluency, TMT A, TMT B, TMT B-A. Scores >2 were considered pathological; d=Raw scores <10 on the CLOX test are considered pathological.

### Example 3

Video-oculography findings

For typical ocular motor findings in an NPC1 heterozygote (subject #7) see FIG. 2A-D. Briefly, reflexive vertical and horizontal saccades were abnormal in NPC heterozygotes compared to healthy controls. To elaborate, in the vertical plane, reflexive saccade duration and duration to reach the peak velocity (duration max) were shorter in heterozygotes for downward saccades in response to 10° and 20° stimuli (duration: p<0.01, duration max: p<0.05), FIG. 3A. This was also the case for latency of downward vertical saccades (p<0.05), FIG. 3A. In the horizontal plane, reflexive saccade duration was shorter in heterozygotes in response to 30° stimulus to the left (p=0.001) and duration max was significantly shorter both right—(p<0.001) and leftward (p<0.01), compared to normal subjects, FIG. 3B (for overview, see Tables 3A-3C).

NPC heterozygotes also displayed abnormal self-paced saccades, both in the vertical and horizontal dimension. In contrast to reflexive saccades, they showed prolonged mean downward saccade duration (vertical, p<0.001; horizontal, p<0.01), FIG. 3C The average peak velocity of horizontal self-paced saccades was 425.9±98.1°/s in mutation carriers and 478.8±85°/s, thus reduced in the mutation carriers (p<0.05).

TABLE 3A

| | Peak velocity, | Amplitude | Latency | Duration | Duration |
|---|---|---|---|---|---|
| (n = 36) | [°/s] | [°] | [s] | [s] | Maximal [s] |
| | Vertical saccades Down | | | | |
| NPC1 Heterozygotes | 398.4 ± 88.6 | 20.1 ± 1.4 | 0.2 ± 0.11 | 0.14 ± 0.04 | 0.06 ± 0.02 |
| Healthy controls | 435.3 ± 82.6 | 20.1 ± 2.1 | 0.2 ± 0.04 | 0.15 ± 0.06 | 0.06 ± 0.02 |
| p-value | NS | NS | <0.05* | <0.01** | <0.05* |
| | Horizontal saccades Right | | | | |
| NPC1 Heterozygotes | 486.6 ± 95.96 | 28.4 ± 2.1 | 0.19 ± 0.05 | 0.18 ± 0.08 | 0.05 ± 0.01 |
| Healthy controls | 512 ± 88.5 | 29 ± 2.3 | 0.2 ± 0.05 | 0.22 ± 0.095 | 0.07 ± 0.02 |
| p-value | NS | NS | NS | NS | <0.001*** |

TABLE 3B

| | Peak velocity, | Amplitude | Latency | Duration | Duration |
|---|---|---|---|---|---|
| (n = 36) | [°/s] | [°] | [s] | [s] | Maximal [s] |
| | Vertical saccades Up | | | | |
| NPC1 Heterozygotes | 406.5 ± 396.3 | 18.4 ± 2.1 | 0.177 ± 0.029 | 0.146 ± 0.065 | 0.05 ± 0.01 |
| Healthy controls | 396.3 ± 66.8 | 18.5 ± 1.7 | 0.194 ± 0.041 | 0.204 ± 0.096 | 0.06 ± 0.024 |
| p-value | NS | NS | NS | NS | NS |
| | Horizontal saccades Left | | | | |
| NPC1 Heterozygotes | 468.5 ± 92.1 | 28.4 ± 2.9 | 0.178 ± 0.039 | 0.163 ± 0.06 | 0.056 ± 0.02 |
| Healthy controls | 478.2 ± 82.2 | 29.1 ± 2.6 | 0.186 ± 0.047 | 0.23 ± 0.1 | 0.073 ± 0.025 |
| p-value | NS | NS | NS | <0.001*** | <0.01** |

TABLE 3C

| | Peak velocity, | Amplitude | Latency | Duration | Duration |
|---|---|---|---|---|---|
| (n = 36) | [°/s] | [°] | [s] | [s] | Maximal [s] |
| | Mean vertical saccades | | | | |
| NPC1 Heterozygotes | 402.5 ± 78.9 | 19.3 ± 1.6 | 0.186 ± 0.058 | 0.144 ± 0.048 | 0.055 ± 0.01 |
| Healthy controls | 415.8 ± 61.7 | 19.6 ± 1.6 | 0.199 ± 0.037 | 0.177 ± 0.068 | 0.062 ± 0.2 |
| p-value | NS | NS | NS | <0.05* | 0.057 |
| | Mean horizontal saccades | | | | |
| NPC1 Heterozygotes | 477.6 ± 90 | 28.4 ± 2.1 | 0.18 ± 0.04 | 0.172 ± 0.067 | 0.053 ± 0.012 |
| Healthy controls | 495.3 ± 83.3 | 29.1 ± 2.1 | 0.2 ± 0.04 | 0.224 ± 0.092 | 0.069 ± 0.02 |
| p-value | NS | NS | NS | <0.01** | <0.001*** |

The duration of self-paced saccades was longer than that of reflexive saccades, both in NPC heterozygotes and controls (p<0.001; p<0.001). Finally, both smooth pursuit and horizontal vestibulo-ocular reflex (VOR gain 0.93) were within the 95% CI for normal subjects.

Regarding gaze-holding, in heterozygotes the vertical and horizontal component of gaze-holding nystagmus on upgaze was higher than in healthy controls (p<0.05), FIG. 3D. Moreover, slow-phase velocity of gaze-holding nystagmus to the right was also higher than in healthy controls (p<0.01).

### Video-Oculographic Instructions:

Reflexive Saccades: Subjects made visually guided vertical and horizontal saccades in response to stimuli of 1.33° visual angle (expression changing smileys). Vertical saccades, elicited by the stimuli of 10° and 20° amplitude over the range ±10° from the central position and horizontal saccades of 15° and 30° amplitude, over the range ±150 from the central position, were required. Participants performed seven saccades in response to the stimulus of each size along both axes. The targets were presented in pseudorandom order for the time of 2500 ms with additional variation of 500 ms.

Self-paced saccades: Subjects were instructed to switch arbitrarily between presented visual stimuli as quickly as possible (“make it a race”). Visual stimulation were permanently visible two dots at 10° up and down, with stimulus amplitude of 20° in the vertical plane. Horizontally, there were two permanently visible dots at 15° on the right and left side, with stimulus amplitude of 30°. Test duration yielded 30 sec. Intersaccadic interval and total number of performed saccades, were evaluated.

Smooth pursuit: After the initial fixation period of 2 s, the target subtending visual angle of 0.57° moved in 3 cycles at 0.1 and 3 cycles at 0.2 Hz frequencies, yielding peak target velocities of 9.5°/s, 18.8°/s horizontal and 6.4°/s, 12.6°/s, with ±15° amplitude horizontally (right and left) from the central position, and then ±10° vertically (up and down) from the central position without a break.

Gaze-holding: Participants were asked to foveate a point of 0.57° visual angle, presented on a monitor without moving the head. The point was primarily positioned at the level of the eyes, then changing the eccentricity from 15° left to 15° right (altogether 30° horizontally) and 10° down. Stimulus was presented for 10 s at each position.

Vestibulo-ocular reflex: Participants were instructed to foveate a white point of visual angle of 0.3° positioned 2 m from the wall. Ten f two head impulses were performed to each side.

Ocular motor data of heterozygotes were compared with the age- and sex-matched heathy controls.

### Example 4

### Imaging Results

¹⁸FDG-PET examination was performed in 16 heterozygotes. Visual rating of stereotactic surface projections by two experiences nuclear medicine physicians revealed significant hypometabolism patterns in 8/16 cases (FIG. 4). In case of disagreement a consensus was achieved. Subregions rated positive for hypometabolism were the cerebellum (8/16), anterior cingulate cortex (8/16), parieto-occipital (8/16) and temporal (5/16). Significant hypometabolism was more likely at older age as assessed by averaged ages of binarized negative or positive rates subjects (neg: 47.8y, pos: 60.0y, p=0.012). Hypometabolism also negatively correlated with education years.

Voxel-based analysis compared to age-matched controls indicated decreased metabolic rates in large clusters of the bilateral cerebellum (−8.8%), bilateral inferior temporal gyrus (−8.0/6), the left anterior cingulate cortex (−9.4%), and the left parietal lobe (−10.8%) (FIG. 5), Smaller clusters of increased brain metabolism were detected in midbrain and tegmentum, right insula, followed by bilateral postcentral region, left insula, and posterior cingulate cortex (in the abovementioned order: 15.5%, 7.9%, 6.8%, 6.4% and 5.5%).

Abdominal ultrasound revealed organomegaly of liver and/or spleen of mild or medium-degree in 10 out of 14 participants, who underwent the examination.

### Example 5

### Correlations

Blood Parameters with Ocular Motor Function, Ultrasound and PET Findings

Cholestantriol concentrations were positively related to the duration of upward and downward saccades, (up: ρ=0.720, p<0.001; down: ρ=0.679, p<0.002), duration to reach the peak velocity of upward saccades (duration max up: ρ=0.598, p<0.01), as well as duration of saccades to the right (ρ=0.657, p<0.01). This was also the case for chitotriosidase activity and duration and duration max of vertical saccades (ρ=0.630, p<0.01, ρ=0.529, p<0.05). The volume of liver and spleen (ρ=0.592, p<0.01) and metabolic activity of the right insula (ρ=0.568, p<0.05) were also positively related to cholestantriol concentration.

### Motor Function and Cognitive Measures

The clinical motor score was related to the second round of re-called words (ρ=0.636, p<0.01). Parameters of the grooved pegboard task positively correlated with domains of CERAD battery, in particular with the memory recall (PH: ρ=0.696, p<0.01; NP: ρ=0.639, p=0.01; both hands: ρ=0.533, p<0.05; PEG assembly: ρ=0.687, p<0.01), but also with the full score of executive function (ρ=−0.512, p<0.05), S-words (ρ=0.546, p<0.01), word list recall 3^{rd} round (ρ=0.577, p<0.05), direct recall (ρ=0.696 p<0.01); and correlated negatively to z-values of intrusions (ρ=−0.640, p<0.01), and discrimination recall (ρ=−0.532,p<0.05).

### Motor Performance and Ocular Motor Function

The clinical motor score was negatively related to mean horizontal saccadic duration, especially to the left (both sides: ρ=−0.480, p<0.05; left: ρ=−0.479, p<0.05). The grooved pegboard test and smell test did not significantly correlate with ocular motor measures.

### Ocular Motor Function and Cognitive Performance

Duration of upward saccades was positively related to discrimination recognition (ρ=0.578, p<0.05). Duration to reach the peak velocity of upward (ρ=−0.548, p<0.05) and leftward saccades (ρ=0.631, p<0.05) were related to word list discrimination recall.

Peak velocity of horizontal saccades also correlated with constructional copying (ρ=0.555, p<0.05) but was negatively related to percentage of savings (ρ=−0.692, p<0.01). The normative age-matched database-corrected savings (z-value) of savings' percentage also negatively correlated with velocity of horizontal saccades (ρ=−0.679, p<0.01), but not of vertical saccades.

Vertical smooth pursuit correlated with percentage of savings (ρ=0.613, p<0.05). Z-value of percentage of savings was negatively related to horizontal smooth pursuit and vertical gain (ρ=−0.579, p<0.05; ρ=−0.521, p<0.05). Both vertical (ρ=0.623, p<0.05; ρ=0.550, p<0.05) and horizontal (ρ=0.826, p<0.0001; ρ=0.779, p<0.001) smooth pursuit correlated with constructional copying tasks including its z-values.

### Brain Metabolism and Motor, Cognitive and VOG Measures

Glucose metabolic rates in the brainstem, but not in postcentral and/or cerebellar areas were negatively correlated with the motor score (ρ=−0.614, p<0.01). Of the cognitive parameters, the BNM test z-value showed a positive relationship with the left insula (ρ=0.609, p<0.05). Intrusions were related to left ACC (ρ=−0.597, p<0.05) and right temporal areas (ρ=−0.615, p<0.05). Posterior cingular cortex was negatively associated with duration until maximal velocity of vertical saccades (ρ=−0.535, p<0.05). Postcentral area correlated negatively with duration of horizontal saccades to the left (ρ=−0.642, p<0.01). Left parietal region correlated positively with (ρ=0.561, p<0.05) peak velocity of horizontal saccades.

### Example 6

### Discussion of Results

Twenty asymptomatic heterozygote NPC1 gene mutation carriers were studied to identify potential early signs of neurodegeneration using a combined approach of different methods (e.g. clinical assessment, cognition testing, ocular motor examination, imaging of visceral organs and brain function, blood analysis, etc.). The test battery was deliberately chosen to detect early stages of neurodegeneration, e.g. PD or dementia (Postuma et al. 2016).

The main findings are as follows. First, NPC heterozygotes recapitulate characteristic features of symptomatic NPC disease: NPC heterozygotes demonstrated oculomotor abnormalities with prolonged duration of saccades, horizontally more than vertically, as well as abnormal horizontal self-paced saccades. There was also the presence of hepatosplenomegaly and increased levels of blood biomarkers characteristic of NPC disease.

Second, NPC heterozygosity is associated with late-onset neurodegeneration. The clinically asymptomatic mutation carriers showed impaired age-corrected cognitive performance, especially affecting visuo-constructive function, verbal fluency and executive function. PET imaging revealed significant abnormal glucose metabolic rates in half of 16 subjects scanned, including one with bilateral abnormalities. This argues for functional consequences of clinically silent heterozygous gene variations.

Third, two novel intronic mutations in the NPC1 gene are described.

Impaired vertical saccades due to supranuclear involvement, later in the disease course also gaze palsy (with combined impairment of smooth pursuit) are a leading symptom of NPC disease. Reflexive and self-paced saccadic duration and reflexive saccades duration to reach the peak velocity are sensitive markers that allow discrimination between NPC1 heterozygotes and healthy controls. Previous studies found that intersaccadic intervals and the absolute number of performed cognitively-driven saccades as good biomarkers to track NPC disease progression (Abel et al. 2009). This study did not find abnormalities of these markers. But prolongation of duration was found in the cohort of clinically asymptomatic NPC heterozygote mutation carriers. Since both peak velocity and duration are driven by burst neurons in the brainstem, prolongation of self-paced saccades might be the earliest component of pathognomonic vertical supranuclear saccade palsy in NPC patients. The discrepancy between the duration of reflexive (shorter than controls) and self-paced saccades (longer than controls) is not completely understood. Without wishing to be bound by any particular theory, prolongation of self-paced saccades might reflect impaired cognition and processing speed in NPC heterozygotes due to the functional disturbance of frontal eye field (FEF). In contrast to NPC patients, horizontal self-paced saccades in heterozygotes were more affected than vertical ones, pointing to a possible additional disturbance of saccadic burst neurons in pons, not midbrain. This is aligned with the brain metabolism analyses.

Vertical saccade duration and maximal duration of rightward horizontal saccades correlated consistently with the volume of left temporal area. The strongest relationships showed abnormalities of upward, followed by downward saccade duration. This is in line with the increase of more than 15% in midbrain metabolic rates, where the vertical saccadic burst neurons are located in the rostral interstitial nucleus of medial longitudinal fascicle (riMLF), thus explaining the short latency and duration of downward vertical saccades. The underlying mechanisms are not fully understood. Without wishing to be bound by any particular theory, there may be paradoxical over-activation in order to compensate for presumable underlying functional deficits. This may be a mechanism similar to vestibular compensation after a unilateral vestibular loss, when the activation is modulated during the recovery process to compensate for the functional deficits (Dieringer et al. 1995; Brandt et al. 1997). No peripheral vestibular impairment in heterozygotes as reflected by intact horizontal vestibulo-ocular reflexes was found similar to findings in homozygous patients with NPC disease (Bremova et al. 2016).

Recent studies showed a specific pattern of brain metabolic abnormality in patients with NPC disease: bilateral hypometabolism in the frontal, prefrontal cortex and bilateral parietotemporal regions, and hypermetabolism in the parietal-occipital white matter, lenticular nucleus of the basal ganglia, cerebellum and pons, which correlated with disease progression (Benussi et al. 2015; Huang et al. 2011; Kumar et al. 2011). Hypometabolism in frontal and parieto-temporal cortices of NPC heterozygotes was found but the most obvious cluster of hypometabolism was in the cerebellum. The cerebellar hypometabolism (which is often severely affected in NPC disease and also plays a key role in saccadic regulation) did not show any significant relationship with any of the investigated ocular motor parameters, especially not smooth pursuit.

Hepatomegaly and splenomegaly are core features of NPC. Indeed, NPC is recognized as a significant cause of liver disease in early life. In those with later onset, it often (50-90%) remains asymptomatic and unrecognized clinically (Patterson et al. 2013). Consistent splenomegaly in heterozygous gene mutation carriers has also been reported (Harzer et al. 2014). In the studied cohort, five participants (more than one third) had organomegaly, two of whom presenting with isolated splenomegaly. If present, particularly when combined with neurodegenerative or psychiatric features, this is highly suggestive of NPC (Patterson et al. 2012). One of the two subjects with isolated splenomegaly also screened positive for REM sleep behavior disorder (RBD). RBD and olfactory loss are markers for which there is now very strong evidence that they have the ability to predict conversion to PD (Postuma et al. 2016).

Finally, levels of NPC biomarkers chitotriosidase and/or cholestantriol, were increased in some of the NPC heterozygotes.

This testing battery was deliberately chosen to include markers of early PD including hyposmia. Four participants (20% of subjects) had a reduced sense of smell, one of whom also had abnormal chitotriosidase activity levels. The ‘Sniffin Sticks’ (Hummel et al. 1997) used here enable testing of three aspects of smell, i.e. smell identification, threshold and discrimination. A reduced sense of smell is associated with Lewy body pathology affecting the olfactory bulbs and cortex (Hoyles et al. 2013). However, as olfaction also depends on an intact memory, impairment may also be present in degenerative non-synucleinopathies associated with cognitive impairment where disruption of specific associative areas involved in olfactory processing may play a role, e.g. vascular dementia, AD and FTLD (Olichney et al. 2005; Gray et al. 2001; Orasji et al. 2016).

Psychometric testing using the CERAD battery was also performed. This revealed impaired overall cognitive function with deficits of visuo-constructive abilities and verbal fluency, as well as impaired executive function (i.e., processing speed, set-shifting), especially in heterozygotes #1, 6, 8 and 14, albeit there was no relationship between brain metabolism, especially frontal and temporo-parietal lobes, and cognitive scores.

To compare, in symptomatic NPC disease cognitive dysfunction manifests as aphasia, apraxia, memory impairment and deficits of executive functions and attention (Heitz et al. 2017; Sevin et al. 2007), particularly in those who presented with a “dementia plus” syndrome (Cupidi et al. 2017) with underlying frontal hypometabolism (Huang et al. 2011; Kumar et al. 2011; Battisti et al. 2003). However, given a relatively preserved verbal episodic memory in NPC patients, compared to Alzheimer disease, it was recently concluded that delayed verbal memory recall may be the best distinguishing factor between the two disorders (Johnen et al. 2018). Thus, the pattern of NPC heterozygotes in this study is in line with the findings in symptomatic NPC disease.

In this study, NPC mutation carriers impaired executive function also manifested as prolonged duration of self-paced saccades seen carriers which have a considerable cognitive component controlled by the frontal eye field (Leigh et al. 2015). Self-paced saccades are frequently impaired in PD (Pretegiani et al. 2017) and dementia including frontotemporal variants (Douglass et al. 2018).

Lastly, two novel variations in intronic regions of NPC1 gene have been identified. The first, c.3246-25A>G is located in a conserved NPC1 cysteine-rich domain and might impact binding motives for splicing regulatory elements (SRP) proteins, which can lead to the inclusion or exclusion of exons (Greer et al. 1999). Findings were confirmed by the translated RNA. The mutation segregated in the family with the disease, where both parents (consanguine marriage) are heterozygous and both affected children are homozygous. The second, c.3246-5_3246-7delx, results in a 3 bp intronic deletion with skipping of exon 22. Neither variation was detected by routine genetic testing.

### Example 7

### NPC Heterozygote Studies

Heterozygous carriers of NPC1 that are either asymptomatic or showing preclinical symptoms of NPC disease, e.g., oculomotor abnormalities, hepatosplenomegaly, elevated levels of chitotriosidase in blood or plasma, and/or elevated levels of cholestane-3β,5α,6β-triol in blood or plasma, will be identified and treated with 5-15 g/day of acetyl-leucine for one to thirty six months. These subjects will be assessed for quality of life (function), gait, and/or ocular motor function before initiating treatment with acetyl-leucine and at regular intervals during the treatment cycle. The subjects may also undergo neurological evaluations and their blood or plasma may be analysed for chitotriosidase activity and cholestane-3β,5α,6β-triol concentration during the treatment cycle. ¹⁸FDG-PET examinations may also be performed before, during, or after treatment with acetyl-leucine. Administration of acetyl-leucine is expected to benefit heterozygous carriers of NPC1 who are at risk of developing late-onset neurodegenerative disease. These benefits include, but are not limited to, improved cognitive function, e.g., as determined by CERAD, decreased levels of chitotriosidase and/or cholestane-3β,5α,6β-triol in blood or plasma, improved ocular motor function, e.g., improved reflexive vertical and horizontal saccades, self-paced saccades, and/or gaze-holding, reduced hypometabolism, or reduced organomegaly of liver or spleen.

### REFERENCES

1. Patterson M. Niemann-Pick Disease Type C. In: Pagon R A, Adam M P, Ardinger H H, et al., eds. Gene Reviews®. Seattle (WA), 1993.
2. Kresojevic N, Dobricic V, Svetel M, Kostic V. Mutations in Niemann Pick type C gene are risk factor for Alzheimer's disease. Medical hypotheses 2014; 83:559-562.
3. Sidransky E, Nalls M A, Aasly J O, et al. Multicenter analysis of glucocerebrosidase mutations in Parkinson's disease. N Engl J Med 2009; 361:1651-1661.
4. Josephs K A, Matsumoto J Y, Lindor N M. Heterozygous Niemann-Pick disease type C presenting with tremor. Neurology 2004; 63:2189-2190.
5. Harzer K, Beck-Wodl S, Bauer P. Niemann-pick disease type C: new aspects in a long published family—partial manifestations in heterozygotes. JIMD Rep 2014; 12:25-29.
6. Kluenemann H H, Nutt J G, Davis M Y, Bird T D. Parkinsonism syndrome in heterozygotes for Niemann-Pick C1. Journal of the neurological sciences 2013; 335:219-220.
7. Cupidi C, Frangipane F, Gallo M, et al. Role of Niemann-Pick Type C Disease Mutations in Dementia. J Alzheimers Dis 2017; 55:1249-1259.
8. Zech M, Nubling G, Castrop F, et al. Niemann-Pick C disease gene mutations and age-related neurodegenerative disorders. PLoS One 2013; 8:e82879.
9. Probert F, Ruiz-Rodado V, Vruchte D T, et al. NMR analysis reveals significant differences in the plasma metabolic profiles of Niemann Pick C1 patients, heterozygous carriers, and healthy controls. Sci Rep 2017; 7:6320.
10. Strauss E. A Compendium of Neuropsychological Tests: Administration, Norms, and Commentary. In: Oxford University Press, 2006: 1042 ﬀ.
11. Bremova-Ertl T, Schiffmann R, Patterson M C, et al. Oculomotor and Vestibular Findings in Gaucher Disease Type 3 and Their Correlation with Neurological Findings. Front Neurol 2017; 8:711.
12. Beck A T, Ward C H, Mendelson M, Mock J, Erbaugh J. An inventory for measuring depression. Archives of general psychiatry 1961; 4:561-571.
13. Stiasny-Kolster K, Mayer G, Schafer S, Moller J C, Heinzel-Gutenbrunner M, Oertel W H. The REM sleep behavior disorder screening questionnaire—a new diagnostic instrument. Movement disorders:official journal of the Movement Disorder Society 2007; 22:2386-2393.
14. Hummel T, Sekinger B, Wolf S R, Pauli E, Kobal G. ‘Sniffin' sticks’: olfactory performance assessed by the combined testing of odor identification, odor discrimination and olfactory threshold. Chem Senses 1997; 22:39-52.
15. Ehrensperger M M, Berres M, Taylor K I, Monsch A U. Early detection of Alzheimer's disease with a total score of the German CERAD. J Int Neuropsychol Soc 2010; 16:910-920.
16. Arbuthnott K, Frank J. Trail making test, part B as a measure of executive control: validation using a set-switching paradigm. J Clin Exp Neuropsychol 2000; 22:518-528.
17. Sanchez-Cubillo I, Perianez J A, Adrover-Roig D, et al. Construct validity of the Trail Making Test: role of task-switching, working memory, inhibition/interference control, and visuomotor abilities. J Int Neuropsychol Soc 2009; 15:438-450.
18. Royall D R, Cordes J A, Polk M. CLOX: an executive clock drawing task. Journal of neurology, neurosurgery, and psychiatry 1998; 64:588-594.
19. Luppa M, Riedel-Heller S G, Luck T, et al. Age-related predictors of institutionalization: results of the German study on ageing, cognition and dementia in primary care patients (AgeCoDe). Soc Psychiatry Psychiatr Epidemiol 2012; 47:263-270.
20. Schneider E, Villgrattner T, Vockeroth J, et al. Eye See Cam: an eye movement-driven head camera for the examination of natural visual exploration. Ann NY Acad Sci 2009; 1164:461-467.
21. Reunert J, Lotz-Havla A S, Polo G, et al. Niemann-Pick Type C-2 Disease: Identification by Analysis of Plasma Cholestane-3beta,5alpha,6beta-Triol and Further Insight into the Clinical Phenotype. JIMD Rep 2015; 23:17-26.
22. Beyer L, Meyer-Wilmes J, Schonecker S, et al. Clinical Routine FDG-PET Imaging of Suspected Progressive Supranuclear Palsy and Corticobasal Degeneration: A Gatekeeper for Subsequent Tau-PET Imaging? Front Neurol 2018; 9:483.
23. Brendel M, Schonecker S, Hoglinger G, et al. [(18)F]-THK5351 PET Correlates with Topology and Symptom Severity in Progressive Supranuclear Palsy. Frontiers in aging neuroscience 2017; 9:440.
24. Havla J B, T.; Sztatecsny, C.; Moser, M.; Lotz-Havla, A.; Maier, E.; Schinner, R.; Kümpfel, T.; Strupp, M.; Schneider, S.A. Retinal axonal degeneration in Niemann Pick type C disease and asymptomatic mutation carriers. (in preparation).
25. Postuma R B, Berg D. Advances in markers of prodromal Parkinson disease. Nat Rev Neurol 2016; 12:622-634.
26. Abel L A, Walterfang M, Fietz M, Bowman E A, Velakoulis D. Saccades in adult Niemann-Pick disease type C reflect frontal, brainstem, and biochemical deficits. Neurology 2009; 72:1083-1086.
27. Dieringer N. ‘Vestibular compensation’: neural plasticity and its relations to functional recovery after labyrinthine lesions in frogs and other vertebrates. Prog Neurobiol 1995; 46:97-129.
28. Brandt T, Strupp M, Arbusow V, Dieringer N. Plasticity of the vestibular system: central compensation and sensory substitution for vestibular deficits. Advances in neurology 1997; 73:297-309.
29. Gunther L, Beck R, Xiong G, et al. N-acetyl-L-leucine accelerates vestibular compensation after unilateral labyrinthectomy by action in the cerebellum and thalamus. PLoS One 2015; 10:e0120891.
30. Bremova T, Krafczyk S, Bardins S, Reinke J, Strupp M. Vestibular function in patients with Niemann-Pick type C disease. Journal of neurology 2016; 263:2260-2270.
31. Benussi A, Alberici A, Premi E, et al. Phenotypic heterogeneity of Niemann-Pick disease type C in monozygotic twins. Journal of neurology 2015; 262:642-647.
32. Huang J Y, Peng S F, Yang C C, Yen K Y, Tzen K Y, Yen R F. Neuroimaging findings in a brain with Niemann-Pick type C disease. J Formos Med Assoc 2011; 110:537-542.
33. Kumar A, Chugani H T. Niemann-Pick disease type C: unique 2-deoxy-2[(1)(8)F]fluoro-D-glucose PET abnormality. Pediatric neurology 2011; 44:57-60.
34. Patterson M C, Mengel E, Wijburg F A, et al. Disease and patient characteristics in NP-C patients: findings from an international disease registry. Orphanet J Rare Dis 2013; 8:12.
35. Patterson M C, Hendriksz C J, Walterfang M, Sedel F, Vanier M T, Wijburg F. Recommendations for the diagnosis and management of Niemann-Pick disease type C: an update. Molecular genetics and metabolism 2012; 106:330-344.
36. Scaglione C, Vignatelli L, Plazzi G, et al. REM sleep behaviour disorder in Parkinson's disease: a questionnaire-based study. Neurological sciences: official journal of the Italian Neurological Society and of the Italian Society of Clinical Neurophysiology 2005; 25:316-321.
37. Hoyles K, Sharma J C. Olfactory loss as a supporting feature in the diagnosis of Parkinson's disease: a pragmatic approach. Journal of neurology 2013; 260:2951-2958.
38. Olichney J M, Murphy C, Hofstetter C R, et al. Anosmia is very common in the Lewy body variant of Alzheimer's disease. Journal of neurology, neurosurgery, and psychiatry 2005; 76:1342-1347.
39. Gray A J, Staples V, Murren K, Dhariwal A, Bentham P. Olfactory identification is impaired in clinic-based patients with vascular dementia and senile dementia of Alzheimer type. Int J Geriatr Psychiatry 2001; 16:513-517.
40. Orasji S S, Mulder J L, de Bruijn S F, Wirtz P W. Olfactory dysfunction in behavioral variant frontotemporal dementia. Clinical neurology and neurosurgery 2016; 141:106-110.
41. Heitz C, Epelbaum S, Nadjar Y. Cognitive impairment profile in adult patients with Niemann pick type C disease. Orphanet J Rare Dis 2017; 12:166.
42. Sevin M, Lesca G, Baumann N, et al. The adult form of Niemann-Pick disease type C. Brain: a journal of neurology 2007; 130:120-133.
43. Battisti C, Tarugi P, Dotti M T, et al. Adult onset Niemann-Pick type C disease: A clinical, neuroimaging and molecular genetic study. Movement disorders: official journal of the Movement Disorder Society 2003; 18:1405-1409.
44. Johnen A, Pawlowski M, Duning T. Distinguishing neurocognitive deficits in adult patients with NP-C from early onset Alzheimer's dementia. Orphanet J Rare Dis 2018; 13:91.
45. Leigh J R Z, D.S. The Neurology of Eye Movements. Fifth edition. Oxford University Press, 2015.
46. Pretegiani E, Optican L M. Eye Movements in Parkinson's Disease and Inherited Parkinsonian Syndromes. Front Neurol 2017; 8:592.
47. Douglass A, Walterfang M, Velakoulis D, Abel L. Behavioral Variant Frontotemporal Dementia Performance on a Range of Saccadic Tasks. J Alzheimers Dis 2018; 65:231-242.
48. Patterson M C, Vecchio D, Jacklin E, et al. Long-term miglustat therapy in children with Niemann-Pick disease type C. Journal of child neurology 2010; 25:300-305.
49. Greer W L, Dobson M J, Girouard G S, Byers D M, Riddell D C, Neumann P E. Mutations in NPC1 highlight a conserved NPC1-specific cysteine-rich domain. American journal of human genetics 1999; 65:1252-1260.
50. Yu W, Ko M, Yanagisawa K, Michikawa M. Neurodegeneration in heterozygous Niemann-Pick type C1 (NPC1) mouse: implication of heterozygous NPC1 mutations being a risk for tauopathy. The Journal of biological chemistry 2005; 280:27296-27302.

All of the features described herein (including any accompanying claims, abstract and drawings), and/or all of the steps of any method so disclosed, may be combined with any of the above aspects in any combination, except combinations where at least some of such features and/or steps are mutually exclusive.

It is to be understood that the foregoing embodiments and exemplifications are not intended to be limiting in any respect to the scope of the disclosure, and that the claims presented herein are intended to encompass all embodiments and exemplifications whether or not explicitly presented herein.

All patents, patent applications, and publications cited herein are fully incorporated by reference in their entirety.

1. A method of:
(i) treating, preventing, or delaying the onset of a late-onset neurodegenerative disease;
(ii) treating, preventing, or delaying the onset of one or more symptoms of a late-onset neurodegenerative disease; or
(iii) treating, preventing, or delaying the onset of a late-onset neurodegenerative disease and preventing or delaying the onset of one or more symptoms of a late-onset neurodegenerative disease,
the method comprising administering a total daily dose of from about 0.250 g to about 15 g of acetyl-L-leucine to a human subject in need thereof, wherein the subject is a heterozygous NPC1 gene mutation carrier.

2. The method of claim 1, wherein the subject has any one or more of:
(a) an elevated level of chitotriosidase in blood or plasma;
(b) an elevated level of cholestane-3β,5α,6β-triol in blood or plasma;
(c) oculomotor abnormalities; or
(d) hepatosplenomegaly.

3. The method of claim 1, wherein the subject is asymptomatic for the late-onset neurodegenerative disease at the time of the initial administration of the acetyl-L-leucine.

4. The method of claim 1, wherein the method is for preventing or delaying the onset of the late-onset neurodegenerative disease.

5. The method of claim 4, wherein the method is for delaying the onset of the late-onset neurodegenerative disease.

6. (canceled)

7. The method of claim 1, wherein the late-onset neurodegenerative disease is Niemann-Pick type C.

8. The method of claim 1, wherein the late-onset neurodegenerative disease is Alzheimer's disease.

9. The method of claim 1, wherein the late-onset neurodegenerative disease is amyotrophic lateral sclerosis.

10. The method of claim 1, wherein the late-onset neurodegenerative disease is Parkinson's disease.

11. The method of claim 1, wherein the late-onset neurodegenerative disease is dementia.

12. The method of claim 1, wherein the method is for preventing or delaying the onset one or more symptoms of the late-onset neurodegenerative disease.

13. The method of claim 12, wherein the method is for delaying the onset one or more symptoms of the late-onset neurodegenerative disease.

14. The method of claim 13, wherein the late-onset neurodegenerative disease is Niemann-Pick type C, Alzheimer's disease, amyotrophic lateral sclerosis, Parkinson's disease, or dementia.

15. The method of claim 14, wherein the late-onset neurodegenerative disease is Niemann-Pick type C, and the one or more symptoms comprise cerebellar ataxia dysarthria, dysphagia tremor, epilepsy, vertical supranuclear palsy, sleep inversion, gelastic cataplexy, dystonia, spasticity, hypotonia, ptosis, microcephaly, psychosis, progressive dementia, progressive hearing loss, bipolar disorder, or depression.

16. (canceled)

17. The method of claim 14, wherein the late-onset neurodegenerative disease is Alzheimer's disease, and the one or more symptoms comprise difficulty performing familiar tasks, memory loss, disorientation to time and place, loss of good judgment, problems with abstract thinking, misplacing things, rapid mood swings, sudden and dramatic personality changes, loss of initiative, sleeping longer than usual, or loss of interest in usual activities.

18. (canceled)

19. The method of claim 14, wherein the late-onset neurodegenerative disease is amyotrophic lateral sclerosis, and the one or more symptoms comprise muscle weakness or atrophy, spasticity, trouble swallowing or breathing, cramping, or slurred and nasal speech.

20. (canceled)

21. The method of claim 14, wherein the late-onset neurodegenerative disease is Parkinson's disease, and the one or more symptoms comprise tremor, bradykinesia, muscle stiffness, impaired posture and balance, loss of automatic movements, speech changes, or writing changes.

22. (canceled)

23. The method of claim 14, wherein said late-onset neurodegenerative disease is dementia and the one or more symptoms comprise memory loss, difficulty communicating or finding words, difficulty with visual and spatial abilities, difficulty reasoning or problem-solving, difficulty handling complex tasks, difficulty with planning and organizing, difficulty with coordination and motor functions, confusion, or disorientation.

24. (canceled)

25. (canceled)

26. The method of claim 1, wherein the acetyl-L-leucine is administered to the subject in combination with another therapeutic agent.

27. (canceled)

28. (canceled)

29. The method of claim 1, wherein the NPC1 gene mutation is a c.3246-25A>G mutation or a c.3246-5_3246-7del mutation.

30. (canceled)

31. The method of claim 1, wherein the total daily dose of acetyl-L-leucine administered to the human subject is about 1 g, about 2 g, about 3 g, about 4 g, about 5 g, about 6 g, about 7 g, about 8 g, about 9 g, or about 10 g per day.

32. The method of claim 31, wherein the total daily dose of acetyl-L-leucine is split across two administrations or three administrations to the human subject.
